(19) United States
(12) Patent Application Publication
(10) Pub. No.: US 2012/0129244 A1
(43) Pub. Date: May 24, 2012

(54) SYSTEMS, METHODS AND APPARATUSES FOR DEWATERING, FLOCCULATING AND HARVESTING ALGAE CELLS

(76) Inventors: Michael Phillip Green, Pacheco, CA (US); Nicholas Eckelberry, Los Angeles, CA (US); Scott Fraser, Manhattan Beach, CA (US); Brian Goodall, Los Angeles, CA (US)

(21) Appl. No.: 13/274,094

(22) Filed: Oct. 14, 2011

Related U.S. Application Data
(60) Provisional application No. 61/393,940, filed on Oct. 17, 2010.
(60) Provisional application No. 61/405,200, filed on Oct. 20, 2010.

(51) Int. Cl. C12N 1/12; C12M 1/42

(52) U.S. Cl. 435/257.1; 435/283.1

(57) ABSTRACT

Described herein are systems, methods and apparatuses, for dewatering, flocculating and harvesting microorganisms. In various implementations, at least one intracellular product from the microorganisms is recovered and processed according to methods disclosed herein. In some implementations, an electromagnetic field is applied to an aqueous suspension of microorganisms thereby causing flocculation of the microorganisms. In various implementations, the microorganisms are harvested, such as by causing the microorganisms to flocculate and sink to the bottom of a container. Alternatively, the microorganisms are caused to flocculate and float to the surface of the aqueous medium where they can be recovered by skimming. In other implementations, the microorganisms are lysed as they are passed through a second electromagnetic field to release intracellular products, among other desired components of the microorganisms. In some implementations, the intracellular contents of the microorganisms, as well as cellular mass and debris, are recovered and utilized as useful products.

### RELATED APPLICATIONS

This application claims priority to U.S. Provisional Patent Application Ser. No. 61/393,940 filed Oct. 17, 2010, entitled Methods and Apparatus for Dewatering, Flocculation and Harvesting of Algae Cells, and to U.S. Provisional Patent Application Ser. No. 61/405,200 filed Oct. 20, 2010, entitled Methods and Apparatus for Dewatering, Flocculation and Harvesting of Algae Cells.

### FIELD OF THE INVENTION

The invention relates to the fields of energy and microbiology. In particular, the invention relates to methods and apparatuses for dewatering, flocculating and harvesting algae cells.

### BACKGROUND OF INVENTION

The harvesting of microorganisms, such as algae, including, but not limited to intracellular products of microorganisms show promise as a partial or full substitute for fossil oil derivatives or other chemicals used in manufacturing products such as pharmaceuticals, cosmetics, industrial products, biofuels, synthetic oils, animal feed, and fertilizers. However, for these substitutes to become viable, methods for harvesting the cells, including steps of recovering and processing of intracellular products must be efficient and cost-effective in order to be competitive with the refining costs associated with fossil oil derivatives. Current extraction methods used for harvesting microorganisms, such as algae to ultimately yield products for use as fossil oil substitutes are laborious and yield low net energy gains, rendering them unviable for today's alternative energy demands. Such previous methods can also produce a significant carbon footprint, exacerbating global warming and other environmental issues. These prior methods, when further scaled up, produce an even greater efficiency loss due to valuable intracellular component degradation and require greater energy or chemical inputs than what is currently financially feasible from a microorganism harvest. For example, the cost per gallon for microorganism bio-fuel is currently approximately nine-fold over the cost of fossil fuel.

All living cells, prokaryotic and eukaryotic, have a plasma transmembrane that encloses their internal contents and serves as a semi-porous barrier to the outside environment. The transmembrane acts as a boundary, holding the cell constituents together, and keeps foreign substances from entering. According to the accepted current theory known as the fluid mosaic model (S. J. Singer and G. Nicolson, 1972, incorporated herein by reference), the plasma membrane is composed of a double layer (bi-layer) of lipids, an oily or waxy substance found in all cells. Most of the lipids in the bilayer can be more precisely described as phospholipids, that is, lipids that feature a phosphate group at one end of each molecule.

Within the phospholipid bilayer of the plasma membrane, many diverse, useful proteins are embedded while other types of mineral proteins simply adhere to the surfaces of the bilayer. Some of these proteins, primarily those that are at least partially exposed on the external side of the membrane, have carbohydrates attached and therefore are referred to as glycoproteins. The positioning of the proteins along the internal plasma membrane is related in part to the organization of the filaments that comprise the cytoskeleton, which helps anchor them in place. This arrangement of proteins also involves the hydrophobic and hydrophilic regions of the cell.

Intracellular extraction methods can vary greatly depending on the type of organism involved, their desired internal component(s), and their purity levels. However, once the cell has been fractured, these useful components are released and typically suspended within a liquid medium which is used to house a living microorganism biomass, making harvesting these useful substances difficult or energy-intensive.

In most current methods of harvesting intracellular products from algae, a dewatering process has to be implemented in order to separate and harvest useful components from a liquid medium or from biomass waste (cellular mass and debris). Current processes are inefficient due to required time frames for liquid evaporation or energy inputs required for drying out a liquid medium or chemical inputs needed for a substance separation.

Accordingly, there is a need for a simple and efficient procedure for dewatering microorganisms, such as algae, so that they can be harvested and their intracellular products can be recovered and used as competitively-priced substitutes for fossil oils and fossil oil derivatives required for manufacturing of industrial products.

By this invention, we provide an energy efficient manner of coagulating cells, such as algae cells, and subsequently, in a second step, disrupt or lyse the cell membrane thereby releasing desirable materials, such as oil, proteins and other valuable components of the cells.

### SUMMARY OF THE INVENTION

Described herein are systems, methods and/or apparatuses for concentrating microalgae and extracting lipids from the microalgae. In some implementations, the process includes dewatering and harvesting microorganisms, such as algae cells, using an electromotive force (emf). In various implementations, dewatering is carried out by flocculating the cells using a first electromotive force that does not extract the lipids. In such implementations, water is removed from flocculated cells to form a concentrated algae slurry. The concentrated algae can then be further processed to extract the lipids according to some implementations. For example, lipids can be extracted from the concentrated algae slurry using a second electromotive force that compromises the cells or the cell structure of the microalgae and releases their intracellular lipids. The intracellular lipids are recovered and optionally processed to produce a hydrocarbon product such as, but not limited to, a biofuel or biofuels.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be obvious from the description, as set forth hereinafter.

Unless otherwise defined, all technical terms used herein have the same meaning as commonly understood by one of ordinary skill in the art to which this invention belongs.

Although methods, systems and apparatuses similar or equivalent to those described herein can be used in the practice or testing of the present invention, suitable methods, systems and apparatuses are described below. All publications, patent applications, and patents mentioned herein are incorporated by reference in their entirety. In the case of conflict, the present specification, including definitions, will control. The particular embodiments discussed below are illustrative only and not intended to be limiting.

### BRIEF DESCRIPTION OF THE DRAWINGS

In order that the manner in which the above recited and other features and advantages of the present invention are obtained, a more particular description of the invention will be rendered by reference to specific embodiments thereof, which are illustrated in the appended drawings. Understanding that the drawings depict only typical embodiments of the present invention and are not, therefore, to be considered as limiting the scope of the invention, the present invention will be described and explained with additional specificity and detail through the use of the accompanying drawings in which:

FIG. 1 illustrates a portion of a electro-flocculation device according to one embodiment of the invention;

FIG. 2 illustrates a sectional perspective view of biomass flowing in between the anode and cathode wall surfaces of the device of FIG. 1;

FIG. 3 illustrates a perspective view of the anode and cathode tubes of a flocculation device according to one embodiment of the invention;

FIG. 4 illustrates a perspective sectional view of the device of FIG. 3 and including a spiral spacer in between the anode and cathode tubes;

FIG. 5 is a perspective view of a series of lipid extraction devices of FIG. 3 connected in parallel by an upper and lower manifold;

FIG. 6 depicts a diagrammatic flow chart of a method according to one embodiment of the present invention;

FIG. 7 depicts a general flow diagram illustrating various steps of a process for extracting lipids from microalgae according to one embodiment of the present invention;

FIG. 8 illustrates a lipid extraction apparatus with a flowing liquid medium containing a microorganism biomass being exposed to an electromagnetic field caused by an electrical transfer;

FIG. 9 illustrates an overview of a normal sized microorganism cell in relationship to a secondary illustration of a swollen cell during exposure to an electromagnetic field and electrical charge;

FIG. 10 illustrates the lipid extraction apparatus of FIG. 8 with heat being applied and transferred into the liquid medium;

FIG. 11 depicts a general flow diagram illustrating various steps of a process for extracting lipids from microalgae according to one embodiment of the present invention;

FIG. 12 illustrates a side view of a micron mixer in association with a secondary tank containing a biomass and sequences of developing foam layers generated by a micron mixer;

FIG. 13 illustrates a secondary tank containing the liquid medium and a resulting foam layer capable of being skimmed off the surface of the liquid medium, into a form harvest tank;

FIG. 14 illustrates one embodiments of a method and apparatus (system) as described herein for the harvest to useful substances from an algae biomass involving coagulation and settling of the coagulated mass in a secondary settling tank; and

FIG. 15 illustrates another embodiment of a method and apparatus (system) as described herein for the harvest of useful substances from an algae biomass involving single step extraction.

### DETAILED DESCRIPTION

A description of embodiments of the present invention will now be given with reference to the Figures. It is expected that the present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

The following disclosure of the present invention may be grouped into subheadings. The utilization of the subheadings is for convenience of the reader only and is not to be construed as limiting in any sense.

The description may use perspective-based descriptions such as up/down, back/front, left/right and top/bottom. Such descriptions are merely used to facilitate the discussion and are not intended to restrict the application or embodiments of the present invention.

For the purposes of the present invention, the phrase “A/B” means A or B. For the purposes of the present invention, the phrase “A and/or B” means “(A), (B), or (A and B).” For the purposes of the present invention, the phrase “at least one of A, B, and C” means “(A), (B), (C), (A and B), (A and C), (B and C), or (A, B and C).” For the purposes of the present invention, the phrase “(A)B” means “(B) or (AB)”, that is, A is an optional element.

Various operations may be described as multiple discrete operations in turn, in a manner that may be helpful in understanding embodiments of the present invention; however, the order of description should not be construed to imply that these operations are order dependent.

The description may use the phrases “in an embodiment,” or “in various embodiments,” which may each refer to one or more of the same or different embodiments. Furthermore, the terms “comprising,” “including,” “having,” and the like, as used with respect to embodiments of the present invention, are synonymous with the definition afforded the term “comprising.”

The terms “coupled” and “connected,” along with their derivatives, may be used. It should be understood that these terms are not intended as synonyms for each other. Rather, in particular embodiments, “connected” may be used to indicate that two or more elements are in direct physical contact with each other. “Coupled” may mean that two or more elements are in direct physical or electrical contact. However, “coupled” may also mean that two or more elements are not in direct contact with each other, but yet still cooperate or interact with each other.

As mentioned previously, the invention relates to the fields of energy and microbiology. In particular, described herein are systems, methods and apparatuses for dewatering at least one microorganism, such as algae cells, from an aqueous medium containing the same and for recovering the at least one microorganism, such as algae cells, from the aqueous medium in an energy efficient manner.

According to various embodiments, these systems, methods and apparatuses involve subjecting algae cells to an electrical current (or an electromagnetic field (EMF), which is a pulsed electrical current according to some embodiments) based on the algae cells' ability to be magnetically responsive and electrically conductive due to the uptake of nutrients required for their survival. According to various embodiments, most of the above-referenced nutrients contain conductive minerals and, when digested, are retained within the cells' transmembrane(s). In other words, such nutrients, which are consumed by a microorganism biomass in order to sustain biomass cell growth and reproduction, allow the microorganism bio mass to be electrically conductive and magnetically-responsive. According to such embodiments, most aquatic microorganism cells consist of a transmembrane, which houses the internal membrane components, such as the nucleus, chloroplast, proteins, and lipids, wherein most internal regions are surrounded by an internal liquid mass. Due to cellular composition, when exposed to electrical current, intracellular components orient themselves due to electrical adsorption in some embodiments. Thus, according to at least some embodiments, when pulsed electrical current is provided, there is a rapid expansion of the cell dimensions. However, in some embodiments, during an off electrical phase, intracellular components immediately contract back down in size. Thus, according to some embodiments, when electrical current is pulsed in rapid frequency, intracellular components and their surrounding liquid mass undergo rapid rates of expansion and contraction resulting in a rearrangement of their internal components.

While not limited to either the theory or mechanisms by which the invention is realized, it is believed that the electromagnetic field induces or emphasizes the creation of magnetic poles in the microorganism subjected to such electromagnetic field resulting in the attraction of cells so processed to similar cells, resulting in a coagulation or flocculation of the cells into a larger mass. This effect of coagulation or flocculation permits dewatering and separation of the coagulated/flocculated mass of cells from the aqueous medium in which they had been suspended. The below described embodiments illustrate adaptations of these systems, apparatuses and methods. Nonetheless, from the description of these embodiments, other aspects of the invention can be made and/or practiced based on the description provided below.

Thus, in general, described herein are methods and apparatuses for concentrating microalgae and extracting lipids from the microalgae. In some embodiments, the process includes dewatering and harvesting microorganisms, such as algae cells, using an electromotive force or an emf. In various embodiments, dewatering is carried out by flocculating the cells using a first electromotive force that does not extract the lipids. In such embodiments, water is removed from flocculated cells to form a concentrated algae slurry. The concentrated algae can then be further processed to extract the lipids according to some embodiments. For example, lipids can be extracted from the concentrated algae slurry using a second electromotive force that compromises the cells or the cell structure of the microalgae and releases their intracellular lipids. The intracellular lipids are recovered and optionally processed to produce a hydrocarbon product such as, but not limited to, a biofuel.

Further described herein are methods and apparatuses for dewatering algae using electro-flocculation. According to some embodiments, the flocculation is achieved by flowing an algae slurry through a thin channel while simultaneously applying the electromotive force as mentioned above. Flowing the algae through the emf with a limited gap distance allows efficient flocculation and high throughput. According to various embodiments, the electromotive force and flow rate are selected in combination with the particular composition of the algae slurry to flocculate the algae cells without lysing or otherwise rupturing the cells. After electro-flocculation, a portion of the water can be removed (e.g., by decanting or skimming) to produce a concentrated algae slurry, which can then be used as a feed for a lipid extraction process according to various embodiments.

As indicated briefly above, in various embodiments, the flocculated cells can be separated from water using one or more of several different methods. The particular dewatering method may depend on whether the flocculated cells float or sink. For example, flocculated cells that are denser than the aqueous medium sink and can be separated from a portion of the water by decanting or other suitable technique. Alternatively, in the case where the flocculated cells are less dense than the aqueous medium, the flocculated cells float to the surface of the aqueous medium where they can be recovered by skimming or other suitable technique according to various embodiments.

According to some embodiments, the dewatering using electro-flocculation facilitates subsequent extraction processes by producing a more concentrated microalgae slurry. In one embodiment, for example, the concentration of the slurry can be greater than 1% solids while in other embodiments the concentration is greater than 3% or greater than 4%, typically in a range from 1% to 30% solids by weight. In another embodiment, the solids content can be in a range from 1-12% solids while in other embodiments the solids content can be in a range from 3-10% or 4-7%. These ranges can be useful for preparing a slurry for extraction using an emf according to some embodiments. In other embodiments, the greater concentration while maintaining flowability of the slurry greatly increases the efficiency of the extraction step and can produce a slurry that is optimally suited for extraction using an electromotive force. During extraction, intracellular lipids are separated from cellular mass and debris, both of which can be separately recovered and utilized as useful products and/or further processed.

In various embodiments, the present invention relates to a system that includes a first apparatus configured to perform electro-flocculation to produce a flocculated slurry as discussed above. In some embodiments, the flocculated slurry is in fluid communication with a separation apparatus for separating water from flocculated algae to produce a concentrated microalgae slurry.

Specifically, some embodiments relate to an apparatus for flocculating and dewatering algae. The apparatus can include at least one first electrical conductor that acts as a cathode and a second electrically conductive housing that acts as an anode, the at least one first conductor being disposed within the housing, such that a space is defined between the exterior of the first conductor and an interior of the housing according to some embodiments. The space provides a flow path for the aqueous suspension or slurry of algae. In further embodiments, an electrical power source may be operably connected to the first conductor and the housing for creating a first electromagnetic field or EMF by providing an electrical current that is applied between the first conductor and the housing and the aqueous suspension of algae. In various embodiments, a separation tank in fluid communication and downstream of the first electrical conductor and the housing collects flow from the first electrical conductor and the housing.

According to various embodiments, and due to cellular magnetic polarities, a magnetic response occurs once subject cells transit through or otherwise traverse the circuit described above. Specifically, in some embodiments, magnetic cellular alignment of the cells occurs. According to such embodiments, Magnetic cellular alignment occurs as a result of respective positive and negative polarities of the cells when the same are exposed to the electromagnetic field generated during the electrical on (or pulsed) phase(s). After cellular alignment, according to some embodiments, the EMF continues to create a pulling force on the cells while they absorb the electrical current in a way similar to an electrical capacitor storing voltage. This causes the cell's components to exert a magnetic effect creating magnetic poles in the cell. While not limited to traditional methods of coagulation or flocculation, the electromagnetic field to which the cells are subjected causes the cells to coagulate or flocculate upon leaving the electromagnetic field according to such embodiments. In some further embodiments, upon exiting the electromagnetic field, the aqueous medium having the magnetically polarized cells is conveyed to a secondary tank where the cells coagulate with other, similarly electromagnetically processed, cells to form a larger mass which can either sink to the bottom of the tank and be recovered, or alternatively, can be encouraged to float to the surface of the medium in the tank and be recovered. In various embodiments, these coagulated cells may be subsequently treated, with or without intermediate harvesting, by subjecting the coagulated cells to a second EMF, which may originate from the same, or different, apparatus used to create the original EMF.

In further embodiments, a method may include (i) providing an aqueous slurry of microorganisms, wherein at least 70 wt % of the microorganisms are microalgae; (ii) providing an apparatus having an anode and a cathode that in part form a channel that defines a fluid flow path, the anode and the cathode having a gap with a distance in a range from 0.5 mm to 200 mm; (iii) flowing the aqueous slurry through the fluid flow path and applying an electromotive force across that gap that flocculates the microalgae to yield a flocculated algae slurry; (iv) removing a portion of the water from the flocculated algae slurry to form a concentrated microalgae slurry; and (v) providing the concentrated microalgae to an apparatus or system that extracts intracellular lipids from the algae in the concentrated microalgae slurry.

According to some further embodiments, the concentrated microalgae slurry, or a processed intermediate thereof, is also or alternatively in fluid communication with a lipid extraction apparatus or a lysing apparatus configured to either release or extract intracellular lipids from the microalgae. In such embodiments, the release or extraction of intracellular lipids from the microalgae is accomplished using and emf, or a second emf as suggested above. According to such embodiments, in a second step, the concentrated algae slurry is processed to extract the lipids. For example, lipids can be extracted from the concentrated algae slurry using a second electromotive force that compromises the cells, or the structure of the cells, of the microalgae and releases the intracellular lipids contained therein. In further embodiments, as mentioned briefly above, the intracellular lipids are recovered and optionally processed to produce a hydrocarbon product such as, but not limited to, a biofuel or various biofuels. Again, during extraction, intracellular lipids are separated from cellular mass and debris, both of which can be separately recovered and utilized as useful products and/or further processed.

As mentioned above, in various embodiments, the systems and methods or the present invention make use of a lysing apparatus or a lipid extraction apparatus that includes an electrical circuit to produce an EMF. An example of a suitable apparatus that can be used to extract lipids from microalgae can be found in Applicant's co-pending PCT patent application serial number PCT/US2010/031756, the entire disclosure of which is hereby incorporated herein by reference. According to some embodiments, the lysing apparatus may include an outer anode structure (e.g., tube) which provides containment for an inner structure (e.g., electrical conductor) having lesser dimensions than the outer anode structure, the inner structure serving as a cathode. According to further embodiments, a spiral surface, such as one groove; or a plurality of grooves separated by at least one land, much as in the nature of “rifling” in the barrel of a gun, or alternatively, where at least a portion of one or both the inner structure and the outer surface have been removed to create a spiraling surface; or at least two spiraling grooves separated by at least one land on each of the inner structure and the outer surface, is provided. As an alternative, in some embodiments, an electrically insulative, isolator or spacer in parallel to both structures (e.g., the outer tube and internal conductor) provides a liquid seal and provides spacing between the anode and cathode circuits which facilitates uniform electrical distribution and prevents short circuiting of the flow path for the aqueous solution containing the microorganism, such as algae cells.

According to further embodiments, the outer anode structure (e.g., tube) typically includes a pair of containment sealing end caps with one end cap having an entry provision used to accept an incoming flow of microorganism biomass, referred to herein as a live slurry or aqueous suspension including microorganism cells, and an opposing end cap through which the transiting flow of biomass exits. In some embodiments, the inner structure or cathode (e.g., an electrical conductor which may optionally also be a tube of the same or different shape as the outer tube) also typically includes sealed end caps to disallow a liquid flow through the center of the structure (e.g., the inner tube) and to divert the flow into the annulus defined between the wall surfaces of the anode and cathode circuits.

In yet further embodiments, a spiraling isolator or spacer serves as a liquid seal between the two wall surfaces of the electrical conductors and with the thickness of the spacer preferably providing equal distance spacing between the two individual wall surfaces. In some embodiments, the proper spacing can allow for a complete three hundred and sixty degree transfer of electrical current around each circuit assembly and prevent short circuits via contact between the anode and cathode surfaces. In further embodiments, the spiraling isolator may provide a gap between the two wall surfaces allowing a passageway or annulus for a flowing biomass. In various embodiments, the spiraling directional flow provided by the spiraling isolator or spiral surface also provides longer transit duration for greater electrical exposure to the flowing biomass thus increasing the residence time that the biomass is subject to the electrical current thereby improving efficiency in allowing a lower watt per hour consumption rate when the circuit is scaled up in size for large volume flows.

According to various embodiments, a method for extracting lipids from microalgae, includes (i) providing an aqueous slurry of microorganisms, wherein at least 70 wt % of the microorganisms are microalgae; (ii) providing an apparatus having a channel formed from a gap between an inner tube and an outer tube, the inner tube and outer tubes serving as a cathode and an anode for applying an emf across the gap, the channel providing a fluid flow path for the aqueous slurry; (iii) flowing the aqueous slurry through the fluid flow path and applying an electromotive force across that gap that flocculates the microalgae to yield a flocculated algae slurry; (iv) removing a portion of the water from the flocculated algae slurry to form a concentrated microalgae slurry; and (v) providing the concentrated microalgae to an apparatus or system that extracts intracellular lipids from the algae in the concentrated microalgae slurry.

According to additional embodiments, pulsed frequency transfer is conducted on the negative side of the circuit thus being transmitted through the anode with negative transfer to the cathode. This method allows a greater efficiency in electrical energy transfer between the anode and cathode surfaces.

According to some embodiments of either the systems and/or the methods described previously, regulation of the electrical energy applied to the microorganism(s), such as algae cells, can be controlled by adjusting at least one of voltage, current, residence time, pulsation frequency and combinations thereof, so that the microorganism(s) is/are subjected to the electromagnetic field in a manner sufficient to achieve the desired effect of either flocculating and/or dewatering the microorganism(s) or lysing the microorganism(s), such as algae cells. In this way, according to such embodiments, the microorganisms can be efficiently dewatered, flocculated and subjected to extraction with an efficient use of external energy, which can be accurately controlled and adjusted to create the desired effect or degree of dewatering, flocculating and/or harvesting. In some additional embodiments, control can be affected manually. In other embodiments, however, the control can be affected automatically with the aid of a computer to facilitate effectively scaling the relevant process(es) up to an industrial viable dewatering, flocculation and recovery process. In various embodiments, suitable sensors can be used to measure the rate and/or amount of dewatering, flocculation and/or lysing and provide output signals to the computer to control the variables of voltage, current, pulsation frequency and residence time that the cells are subjected to one or more electromagnetic fields in succession.

According to various embodiments, electrical input frequency rates are determined by biomass density with pulse rate frequencies being generally increased when a thicker biomass is present. In such embodiments, biomass density is determined by using a formula based on a percentage of grams of biomass present per liter of flowing liquid medium. Thus, the two-step application of EMF to the algae cells, once when virgin and the second time when coagulated, results in efficient energy utilization and reducing and/or preventing degradation of intracellular components according to various embodiments.

According to such embodiments, use of the foregoing formula allows a programmable microprocessor working in conjunction with a series of sensors to assume operational responsibilities. Based on biomass density formulas, according to various embodiments, an automated matrix dictates to the system the prescribed parameters for flow, the amount of electrical input (voltage and current) and the rate or frequency of pulses required for efficient coagulation or flocculation. In some embodiments, this practice further allows greater energy efficiency in larger scale applications.

Accordingly, described herein is an apparatus for dewatering and/or flocculating at least one microorganism, such as algae cells, from an aqueous suspension. In some embodiments, the apparatus includes at least one first electrical conductor that acts as a cathode and a second electrically conductive housing that acts as an anode, the at least one first conductor being disposed within the housing, such that a space or an annulus is defined between the exterior of the first conductor and an interior of the housing, providing a flow path for the aqueous suspension. According to some further embodiments, at least a portion of one or both surfaces of the first conductor and the housing is removable to create a spiral surface, or at least two spiraling surfaces, such as grooves separated by at least one land that reduces or prevents algae cell buildup on or around the first conductor and the housing. According to additional embodiments, an electrical power source is operably connected to the first conductor and the housing for providing a pulsed electrical current that is applied between the first conductor and the housing and the aqueous suspension for coagulating or flocculating the algae cells resulting in a mass of algae cells that are easy to recover from the aqueous suspension. According to some additional embodiments, a secondary tank, or a separation tank, is operably connected to the first electrical conductor and the housing such that the aqueous suspension can flow from the flow path of the electromagnetic device into the secondary tank for separation of the coagulated or flocculated algae cells from the aqueous suspension. In various embodiments, the first conductor can be a metal tube. In other embodiments, the first conductor and second housing can each be metal tubes, e.g., metal tubes of circular shape, metal tubes of different shapes, etc. In at least one embodiment, the inner diameter of the metal housing and the outer diameter of the first conductor differ in size on the order of 0.050 inches. According to some embodiments, the housing can be a metal tube and the at least one electrical conductor can include a plurality of spaced apart electrical conductors, the electrical conductors being separated from each other by electrically insulating elements; and a multiplicity of flow paths being created between the housing and each of the plurality of spaced apart electrical conductors. In such embodiments, each of the plurality of electrical conductors can be metal tubes.

Also as described herein is a method of recovering the electromagnetically processed algae cells from the aqueous suspension. In some embodiments, the method includes providing an apparatus that includes at least one first electrical conductor that acts as a cathode and a second electrically conductive housing that acts as an anode, the at least one first conductor being disposed within the housing, such that a space or an annulus is defined between the exterior of the first conductor and an interior of the housing, providing a flow path for the aqueous suspension. In various embodiments, at least a portion of one or both surfaces of the first conductor and the housing is removed to create a spiral surface, such as by creating at least one, or at least two, spiral grooves separated by at least one land in at least one of, or both of, the inner and outer surfaces, which spiral surface(s) reduce and/or prevent algae cell buildup on or around the first conductor and/or the housing. In further embodiments, an electrical power source is operably connected to the first conductor and the housing for providing a pulsed electrical current that is applied between the first conductor and the housing and the aqueous suspension for electromagnetically processing the algae cells resulting in a mass of algae cells having magnetic poles thereon or therein to cause the algae cells in the aqueous suspension to coagulate or flocculate. In additional further embodiments, a secondary tank, or a separation tank, is downstream of the first electrical conductor and the housing such that the aqueous suspension can flow in a flow path from the electromagnetic treatment to the secondary tank for separation of the algae cells from the aqueous suspension.

In various embodiments, the method further includes the step of applying a sufficient amount of a pulsed electrical current to the at least one first conductor and the housing and aqueous suspension for causing the effect of coagulating the algae cells once they have left the electromagnetic field, resulting in a mass of coagulated algae cells in the aqueous suspension. In additional embodiments, a step of flowing the aqueous suspension containing the electromagnetically treated cells to the secondary tank for separating the flocculated cells from the aqueous suspension is also contemplated. In such embodiments, the separated cells may optionally be subjected to further processing steps to recover desirable components thereof, such as the intracellular components (including lipids) and the remaining biomass and or debris.

Further described herein is a method of recovering the electromagnetically treated cells from the aqueous suspension including algae cells. In at least one embodiment, for example, the method includes providing an apparatus that includes at least one first electrical conductor that acts as a cathode and a second electrically conductive housing that acts as an anode, the at least one first conductor being disposed within the housing, such that a space or an annulus is defined between the exterior of the first conductor and an interior of the housing, providing a flow path for the aqueous suspension. In a further embodiments, at least a portion of one, or both, surfaces of the first conductor and the housing has been removed to create at least two spiral grooves separated by at least one land that reduces or prevents algae cell buildup on or around the first conductor and the housing. In a further embodiments, an electrical power source is operably connected to the first conductor and the housing for providing a pulsed electrical current that is applied between the first conductor and the housing and the aqueous suspension for electromagnetically processing the algae cells resulting in a mass algae cells that have magnetic poles thereon or therein, coagulating these electromagnetically treated algae cells in the aqueous suspension. In yet another embodiment, a secondary tank, or a separation tank, is connected downstream of the first electrical conductor and the housing such that the aqueous suspension can flow from the flow path into the secondary tank for separation of the at least one of coagulated or flocculated algae cells from the aqueous suspension. According to at least one embodiment, an element is disposed in the secondary tank for producing microbubbles. In some embodiments, an aqueous suspension containing coagulated or flocculated algae cells is disposed in the flow path of the microbubbles and optionally a pump is disposed in the secondary tank for circulating the aqueous suspension. In yet further embodiments, the method further includes the steps of (1) applying a sufficient amount of a pulsed electrical current to the at least one first conductor and the housing and aqueous suspension for coagulating or flocculating the algae cells, (2) flowing the aqueous suspension containing the electromagnetically treated algae cells to the secondary tank in the aqueous suspension, (3) activating the pump and the element for producing microbubbles resulting in a plurality of microbubbles that impinge upon the coagulated and flocculated algae cells so as to cause such cells to float upwards in the aqueous suspension, and (4) separating the floating coagulated and flocculated cells from the aqueous suspension. In various embodiments, the element disposed in the secondary tank for producing microbubbles can be any suitable device or apparatus, e.g. a mixer, a bubble generator or a microbubble generator.

According to various embodiments, in performing the method, a microalgae slurry to be concentrated is harvested and provided. In some embodiments, the microalgae slurry includes water and algae in various proportions. According to various embodiments, the algae cells can be any suitable microalgae cells, including, but not limited to, Nanochloropsis oculata, Scenedesmus, Chlamydomonas, Chlorella, Spirogyra, Euglena, Piymnesium, Porphyridium, Synechoccus sp, Cyanobacteria and certain classes of Rhodophyta single celled strains. In some embodiments, the algae can be phototrophic algae grown in an open natural environment or in a closed environment. In some additional embodiments, the algae is autotrophic algae while in other embodiments, the algae is heterotrophic algae. In some embodiments, the methods of the invention can also be used to concentrate phototrophic, autotrophic and/or heterotrophic algae.

The concentration of the algae in the slurry will depend in part on the type of algae, the growth conditions, available nutrients and other various parameters which may be modified or adjusted according to various embodiments. In other embodiments, such parameters are dictated by nature or the natural environment and the available resources. In some embodiments, the aqueous slurry is grown and used at any suitable concentration, such as, but not limited, to a range from about 100 mg/L to about 5 g/L (e.g., about 500 mg/L to about 1 g/L). In some embodiments, un-concentrated algae from a growth vessel will be from 250 mg/L to 1.5 g/L and may be pre-concentrated with other conventional means to within a range from 5 g/L to 20 g/L.

In at least one embodiment, the algae slurry has a desired concentration of microalgae as a percentage of the total microorganisms in the slurry. In certain embodiments, the purity of the slurry with respect to the concentration of microalgae can impact the composition of the lipids released from the extraction process. Thus, in some embodiments, at least 70 wt % of microorganisms within the aqueous slurry are microalgae while in other embodiments at least 80 wt % of microorganisms within the aqueous slurry are microalgae. In still other embodiments, at least 90 wt % of microorganisms within the aqueous slurry are microalgae while in yet other embodiments at least 95 wt % of microorganisms within the aqueous slurry are microalgae. In still further embodiments, at least 99 wt % of microorganisms within the aqueous slurry are microalgae. According to various embodiments, the aqueous slurry is at least 2% microalgae by weight of the aqueous slurry.

According to some embodiments, the pH of the slurry during flocculation can vary. In various embodiments, the pH is alkaline. However, in other embodiments, acid or base can be added to keep the pH at a desired level or measure, which can be kept in a range from 6.6-9.0, 6.8-8.6, or 7.0-8.5 according to various embodiments.

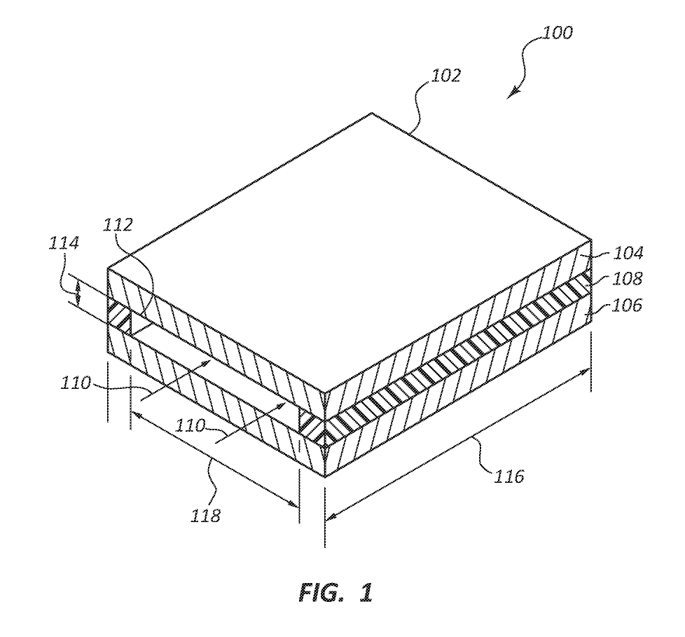

With reference now to the several Figures, the methods, systems and apparatuses described in general previously will be discussed in greater detail with reference to illustrative embodiments. According to various embodiments, some methods include providing a flocculation apparatus that includes, among other things, a fluid path for flowing the algae through an emf that is sufficiently strong to flocculate the cells, but not so strong that the algae cells release their lipids or other intracellular components. In some further embodiments, the apparatus includes an anode and a cathode that form a channel through which the aqueous slurry can flow. For example, FIG. 1 illustrates a schematic of a portion of a flocculation device 100 that is suitable for use in various methods according to some embodiments. In such embodiments, the illustrated portion of flocculation device 100 includes a body 102 that comprises an anode 104 and a cathode 106 electrically separated by an insulator 108. In various embodiments, anode 104 and cathode 106 are spaced apart to form a channel 112 that defines a fluid flow path 110. According to various embodiments, channel 112 has a length 116 that extends the length of the anode and cathode exposed to the fluid flow path 110. Likewise, in various embodiments, channel 112 also has a width 118 that is defined by the space between the insulators 108 that is exposed to the anode 104 and cathode 106. Thus, as illustrated in FIG. 1, and according to some embodiments, channel 112 is bounded on its sides so as to form an opening and an exit through which fluid can be caused to flow (e.g., by pumping). In other embodiments, the methods of the present invention are capable of being carried out without the use of an anode and/or cathode or and anode/cathode pair or without the employment of a device containing an anode/cathode pair.

According to some embodiments, as further illustrated in FIG. 1, the gap 114 between anode 104 and cathode 106 has a distance suitable for applying an emf through the aqueous algae slurry. In at least one embodiment, for example, gap 114 is in a range from 0.5 mm to 200 mm. In various embodiments, gap 114 is in a range from 1 mm to 50 mm while in other embodiments gap 114 is in a range from 2 mm 20 mm. In some embodiments, the narrow gap distance 114 coupled with a comparatively large width 118 and length 116 can provide a large volume for channel 112 while maintaining a strong electrical field for flocculating or otherwise electrically treating the algae cells.

According to some embodiments, the length 116 of channel 112 is the dimension commensurate with or in the direction of fluid flow 110 (the longitudinal direction) and can be any length so long as channel 112 is not occluded by plugging (e.g., with the algae biomass slurry flowing there through) or hampered by significant pressure drops. In at least one embodiment, the length 116 of channel 112 is at least 25 cm. In other embodiments, however, length 116 is 50 cm while in other embodiments length 116 is 100 cm. In still other embodiments, length 116 is at least 200 cm while in yet other embodiments length 116 exceeds 200 cm. In additional embodiments, length 116 can be less than 1000 cm, less than 500 cm or less than 250 cm.

In some embodiments, width 118 of channel 112 can be any width so long as the materials of anode 104 and cathode 106 are sufficiently strong to span the width without contacting one another and thus shorting the system or apparatus. In at least one embodiment, the volume of channel 112 between anode 104 and cathode 106 and within gap distance 114, (i.e., the gap volume) is at least 50 ml. In other embodiments, however, the gap volume is at least 200 ml while in other embodiments the gap volume is at least 500 ml. In yet additional embodiments, the gap volume is at least 1 liter. In other embodiments, the gap volume exceeds 1 liter. In additional embodiments, the surface area of anode 104 and cathode 106 exposed to fluid flow 110 (i.e. the gap surface area) is at least 500 cm². In other embodiments, the gap surface area is at least 1000 cm² while in other embodiments the gap surface area is at least 2000 cm². In yet other embodiments, the gap surface area exceeds 2000 cm².

According to various embodiments, anode 104 and cathode 106 can be made of any electrically conductive material suitable for applying an emf across gap 114, including but not limited to metals such as steel and conductive composites or polymers. In some embodiments, the material selected for anode 104 and cathode 106 are resistant to corrosion while in other embodiments the material selected is non-corrosive.

In various embodiments, the shape of anode 104 and cathode 106 can be planer, cylindrical or any other suitable shape(s). According to at least some embodiments, as described more fully below, an annulus created between an inner conductive (and in some embodiments metallic) surface of a larger tube and an outer surface of a smaller conductive tube (also metallic according to some embodiments) placed within the larger tube is suitable for its ability to avoid fouling and/or shorting and to maintain a high surface area in a compact design. However, the tubes need not have a circular periphery as an inner or outer tube may be square, rectangular, or any other suitable shape according to various embodiments. Moreover, the tube shape does not necessarily have to be the same, thereby permitting tube shapes of the inner and outer tubes to be different in some embodiments. In at least one embodiment, the inner (smaller) conductive tube and outer (larger) conductive tube are concentric tubes, with at least one tube, preferably the outer tube, being provided with a plurality of spiral grooves separated by lands to impart a rifling to the tube. This rifling, according to some embodiments, has been found to decrease build-up of residue on the tube surfaces. In some commercial embodiments, there may be a plurality of inner tubes surrounded by an outer tube to increase the surface contact of the conductors with the algae or to otherwise increase the residence time and/or concentration of electrical current applied to the algae.

In other embodiments, however, the use of electrical insulators, such as plastic tubes, baffles, and other devices, can be used to separate a large flocculation device into a plurality of zones, so as to efficiently scale-up the invention for commercial applications.

With continued reference to FIG. 1, in performing the method according to certain embodiments, the aqueous algae slurry is fed through channel 112 along fluid flow path 110 between anode 104 and cathode 106 (i.e., through the gap 114). According to certain embodiments, power is applied to anode 104 and cathode 106 to produce an electromotive force that flocculates the algae cells without causing the cells to be compromised. In various embodiments, the amount of emf depends on the composition of the aqueous slurry, the gap distance, and the flow rate. However, in certain embodiments, and for a given set of conditions, one can determine whether flocculation is occurring by observing flocculating cells. According to such embodiments, avoiding lysis or cell damage can be determined by observing flocculated cells in a microscope using techniques known in the art.

Thus, disclosed herein are various methods and apparatuses for dewatering algae cells in aqueous suspension or an dewatering an aqueous solution containing algae cells. In some embodiments, the associated systems include a flow path between two surfaces, which in some embodiments are metallic, such as the flow path created between two metal plates of large surface area, separated by a small distance.

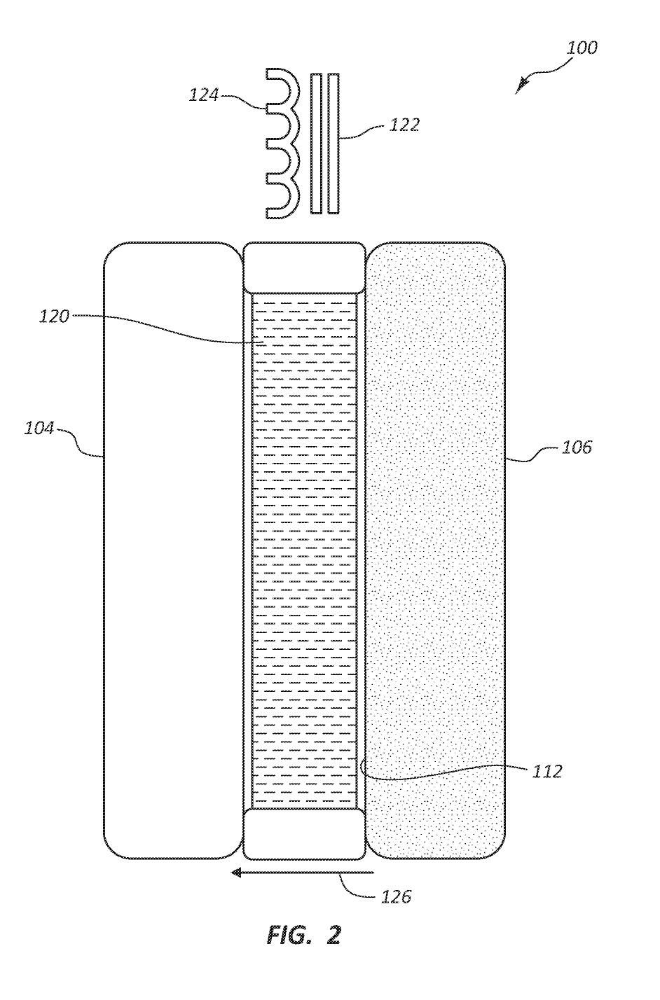

With reference now to FIG. 2, apparatus 100 is shown in cross section with an aqueous algae slurry 120 disposed between cathode 106 and anode 104. According to some embodiments, the aqueous algae slurry 120 is caused to flow through channel 112 using a pump (not shown). In such embodiments, and by way of an electrical conduit, a negative connection 122 is made to the anode 104, which provides electrical grounding. Further according to such embodiments, a positive electrical input 124 is also delivered by way of a conduit connection in order to provide a positive electrical transfer throughout the cathode 106. According to such embodiments, when a positive current 124 is applied to cathode 106, the positive current 124 then seeks a grounding circuit for electrical transfer as indicated by arrow 126, or in this case, to the anode 104, which allows the completion of an electrical circuit. In this respect, and according to such embodiments, a transfer of electrons occurs between the positive and negative surfaces areas but only when an electrically conductive liquid is present between them. Thus, according to such embodiments, as the liquid medium containing the algae slurry 120 is flowed between the surface areas, an electrical transfer from cathode 106 through slurry 120 to anode 104 occurs. In other words, as a liquid containing a microorganism biomass transverses the anode and cathode circuit according to the embodiments described above, the microorganism cells are exposed to an electric field that causes flocculation of the cells or otherwise induces the cells to subsequently flocculate following exposure to the electric field. In some embodiments, as the cells traverse the above-identified circuit, the cells are exposed to both a magnetic field, causing a cellular alignment, and to an electrical field which induces cellular current absorption.

In some embodiments, as mentioned above, the foregoing electrical transfer through the living microorganism is achievable due to nutrients containing electrically conductive minerals suspended within the liquid medium. Accordingly, in various embodiments, the liquid slurry provides a vehicle adapted to impart high electrical transfer through the culture (brine would apply to salt water algae, but the device can successfully process fresh water algae as well) to the algae cells contained therein.

In at least one embodiment, the flow rate through the gap volume (i.e., the portion of channel 112 in the electric field at the gap distance 114) is 0.1 ml/second per ml of gap volume. In other embodiments, however, the flow rate through the gap volume is at least 0.5 ml/second per ml of gap volume while in other embodiments the flow rate through the gap volume is at least 1.0 ml/second per ml of gap volume. In still other embodiments, the flow rate through the gap volume is at least 1.5 ml/second per ml of gap volume. In yet other embodiments, the flow rate through the gap volume exceeds 1.5 ml/second per ml of gap volume. In at least one additional embodiment, the flow rate can be controlled by controlling the pressure using a pump or other suitable fluid flow mechanical devices.

In some embodiments, the emf can be pulsed on and off repeatedly to cause extension and relaxation of the algae cells. According to such embodiments, voltages can be higher and peak amperage lower while average amperage remains relatively low. In such embodiments, this condition or controlled circumstance reduces the energy requirements for operating the apparatus and reduces wear on the anode and cathode pair or pairs. In at least one embodiment, the frequency of the emf pulses is at least about 500 Hz, 1 kHz, 2 kHz, or 30 kHz. In other embodiments, the frequency is less than 200 kHz, 80 kHz, 50 kHz, 30 kHz, 5 kHz, or 2 kHz. Ranges for the pulse frequency can be any combination of the foregoing maximum and minimum frequencies according to various embodiments.

In some embodiments, the power supply provides alternating current while in other embodiments the power supply provides or supplies direct current. Moreover, in some embodiments, the anode and the cathode pair are fixed during flocculation while in other embodiments the anode/cathode pair or circuit alternates during flocculation.

According to various embodiments, the temperature of the aqueous slurry during extraction can also have an impact on the power required to extract the non-polar lipids. Thus, in some embodiments, lipid extraction is carried out at room temperature. However, in at least one embodiment, heat is added to the aqueous algae slurry to achieve a desired temperature. For example, in some embodiments, lipid extraction is carried out at a temperature above 40° F., 65° F., 80° F., 100° F., or 120° F. In other various embodiments, however, the temperature is below 130° F., 115° F., 105° F., or 90° F. while lipid extraction is carried out. According to various embodiments, ranges for the extraction temperature can be any combination of the foregoing maximum and minimum temperatures.

In various embodiments, the average amperage is at least 1 amp, 5 amps, 10 amps, 50 amps, or even at least 100 amps. According to at least some embodiments, the maximum amps can be less than 200 amps, less than 100 amps, less than 50 amps, or less than 10 amps. As with pulse frequency, temperature and the like, the range of amperage can be any range from the foregoing maximum and minimum amperages according to various embodiments.

Similarly, according to various embodiments, the voltage can be at least 1V, 10V, 100V, 1 kV, or even at least 20 kV. In some embodiments, the maximum voltage can be less than 50 kV, less than 30 kV, less than 10 kV, less than 1 kV, or even less than 100V. Again, the range of voltage can be any range of the foregoing maximum and minimum voltages according to various embodiments.

According to embodiments disclosed herein, the foregoing voltages, amperages, pulse frequencies, and flow rates, will depend on various factors such as the power supply, the type of algae, the ions in solution, the temperature, and other similar factors. Nevertheless, those skilled in the art will recognize that flocculation can be easily observed visually. This, in some embodiments, to avoid cell damage and lipid extraction, one can observe the cells under a microscope and/or use the minimum amount of emf that still causes flocculation. Also, according to some embodiments, lower voltage and amperage can be used for given material where flocculation is desired without lysis.

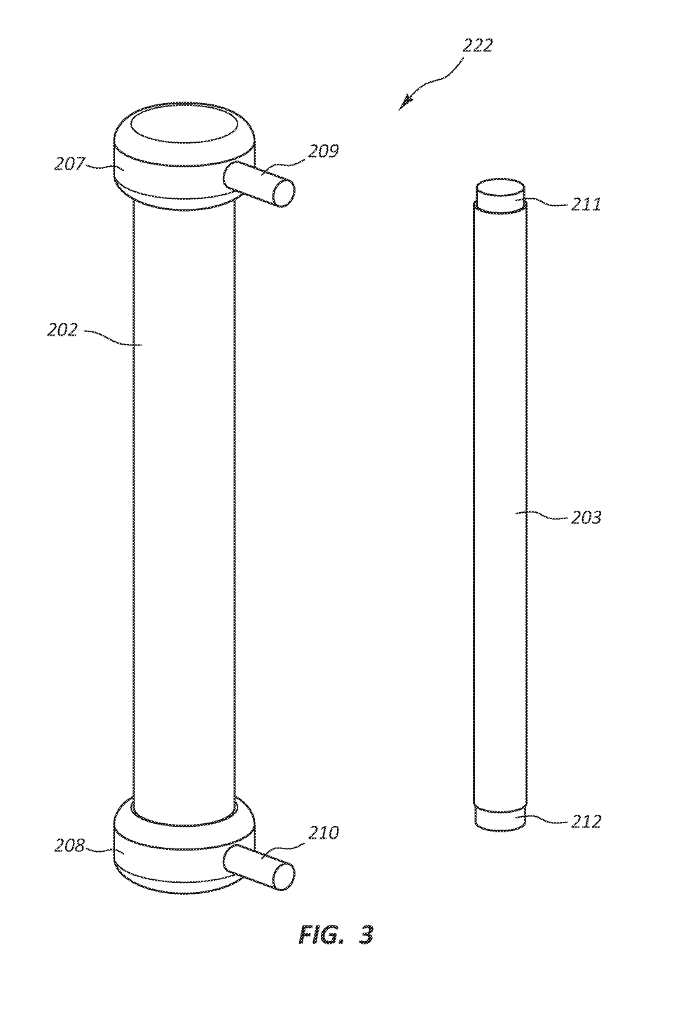

With reference now to FIGS. 3-5, an illustrative lipid extraction apparatus according to some embodiments will be now be described in greater detail. As illustrated in the Figures, the apparatus 222 is comprised of a “tube within a tube” configuration according to some embodiments. According to some embodiments, FIG. 3 illustrates a disassembled flocculation device showing a first (smaller) conductive tube 203 (hereinafter cathode 203, although conductive tube 203 may also be the anode or switch between anode and cathode according to various embodiments) that is configured to be placed in a second (larger) conductive tube 202 (hereinafter anode 202, although conductive tube 202 may also be the cathode or switch between anode and cathode according to various embodiments). In some embodiments, the outer anode tube 202 includes a pair of containment sealing end caps 207 and 208. In such embodiments, sealing end cap 207 provides an entry point 209 used to accept an aqueous algae slurry. Following the transition of the biomass algae slurry through anode 202 according to various embodiments, the opposing end cap 208 provides an exit point 210 to the outward flowing algae biomass.

With continued reference to FIG. 3, in some embodiments, the inner cathode tube 203 includes sealed end caps 211 and 212 to prevent liquid flow through the center of the tube and to divert the flow between the inner surface of anode 202 and the outer surface of cathode 203, thereby forming a channel or annulus between anode 202 and cathode 203. In some embodiments, the channel can be sized and configured as described above with respect to FIG. 1. According to the foregoing embodiments, the use of a “tube within a tube” configuration is particularly advantageous for avoiding fouling or shorting by the algae and/or other microorganisms in the slurry.

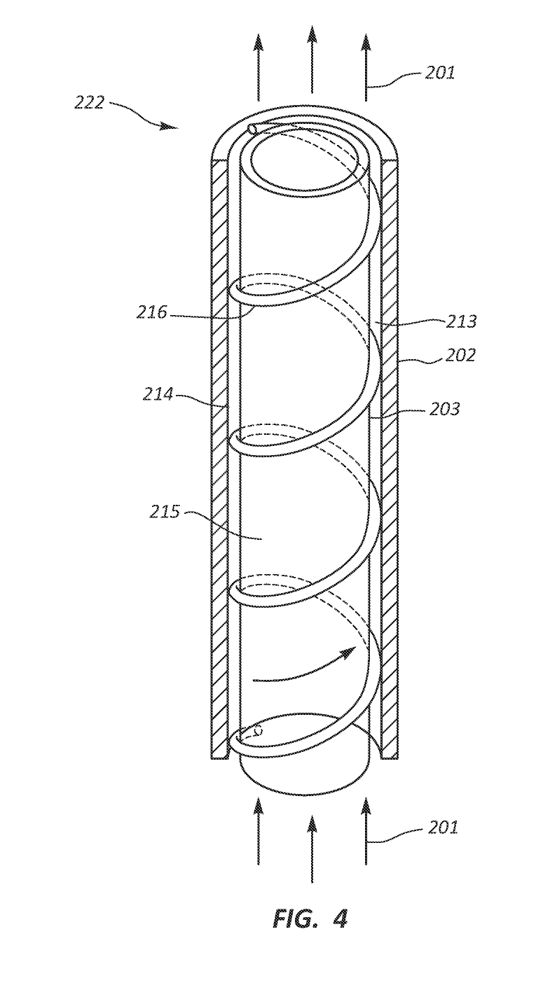

Turning now to FIG. 4, an alternative embodiment of apparatus 222 is illustrated. As seen in FIG. 4, in some embodiments, an insulative spacer 216 is positioned in the channel between anode 202 and cathode 203 to cause spiraling fluid flow. In such embodiments, insulative spiraling isolator spacer 216 serves as a liquid seal between the two wall surfaces 214 and 215 with the thickness of the spacer preferably providing equal distance spacing between anode 202 and cathode 203. According to some embodiments, the spacing and directional flow can cause the fluid flow path to complete a three hundred and sixty degree transfer of electrical current around anode 202 and cathode tube 203. In some further embodiments, the spacer 216 can also help prevent contact between anode 202 and cathode 203, which prevents shorting or fouling the anode and cathode pair and forces electrical current through the liquid medium. According to various embodiments, the spiraling isolator 216 also provides a gap 213 between the two wall surfaces 214 and 215 allowing a passage way for a flowing biomass 201. In some embodiments, the spiraling directional flow further provides a longer transit duration or residence time, which in turn provides greater electrical exposure to the flowing biomass 201 thus increasing flocculation efficiency at a lower per kilowatt hour consumption rate. According to various embodiments, any suitable material can be used as a spacer. According to some embodiments, ceramic, polymeric, vinyl, PVC plastics, bio-plastics, vinyl, monofilament, vinyl rubber, synthetic rubber, or other non-conductive materials are used.

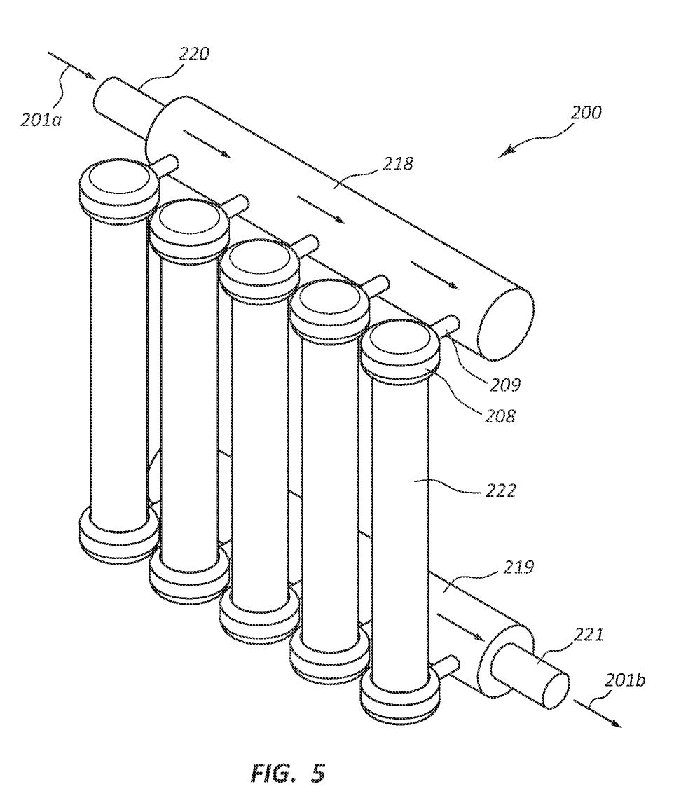

In reference to FIG. 5, a series of anode and cathode circuits 222, according to some embodiments, are shown in parallel having a common upper manifold chamber 218, which receives an in flowing biomass 201a through entry port 220. According to such embodiments, once entering into the upper manifold chamber 218, the biomass 201a makes a downward connection into each individual anode and cathode circuit 222 through entry ports 209, which allow a flowing connection, or a fluid connection, to the sealing end caps 208. According to such embodiments, it is at this point where the flowing biomass 201 (i.e., the aqueous algae slurry) enters into the anode and cathode circuits 222. Further according to such embodiments, once transiting through the individual circuits 222, which in some embodiments comprises a spiral flow path while in other embodiments a strait fluid flow path is contemplated, the flowing biomass 201 exits into a lower manifold chamber 219 where the biomass 201b is then directed to flow out of the apparatus 200 (system) through exit point 221.

In various embodiments, an alternative two-step process of recovering desirable components or intracellular products of the cells, such as algae cells, is contemplated. According to such embodiments, the coagulated cells are subjected to a further EMF following flocculation in order to disrupt or lyse the cell membrane and release at least oil, among other desired components and/or intracellular products of the cells.

According to some embodiments, the various system embodiments discussed above are adjustable, and can be set up with various flow rates, voltage, amperage, electrical pulse frequencies and/or variable temperatures. According to some embodiments, the algae slurry in suspension, after being introduced into the system, enters into an electromagnetic field or EMF and is subjected to a current input for a prescribed transit or residence time (which can be adjusted according to flow rate or through the use of spiraled or rifled circuits 222) which dictates whether the algae biomass is simply electro-coagulated (or flocculated) or if the algae cells are also lysed or ruptured causing a release of intracellular oil. According to some embodiments, various determining factors for this method (selective flocculation with little or no cell rupture) include, but are not limited to:

The amount of energy input (total wattage as a combination of volts and amps);

The frequency in which the electrical input is applied;

The flow length (e.g., a rifled interior circuit 222 can have closer rifle spacing for a longer residence or duration flow or EMF exposure time, or a larger rifle spacing for a shorter duration flow or EMF exposure time);

The electrodes, separated by the rifling, can have a smaller gap for longer duration or EMF exposure time or field strength, or a larger gap for a shorter duration flow or EMF exposure time or field strength;

A combination of the closer/larger rifle spacing, with a larger/smaller electrode gap;

The concentration of the algae culture; and/or

The pH and the salinity of the culture.

According to various embodiments, the longer the total transit or residence time, which can be determined by an adjustable flow rate, in combination with higher electrical input, provides greater EMF exposure to the flowed biomass favoring more efficient algae cell lysing. In some embodiments, when setting lower electrical input and higher flow rate parameters, the system will not lyse or otherwise rupture the cells with the net effect leaving the uncompromised, non-ruptured algae biomass in a total flocculation suspension. According, such embodiments allow greater electrical efficiency and are a much easier process for the separation of the flocculated biomass from their water environment.

According to various embodiments, the parameters discussed and identified herein can be readily tuned by an operator skilled in the art to give the desired result. According to certain embodiments, should any cell rupture be observed in initial settings then the power input can simply be reduced or the exposure time reduced (increasing flow rate) until the desired result is achieved. According to such embodiments, cell rupture can be readily recognized and calibrated using a simple optical microscope with modest magnification (of roughly 40×) common to those of skill in the art.

According to various embodiments, the foregoing flocculation process can be beneficial if the desired outcome does not require the lysing of the cells, but just the biomass being separated from the water as when they were in suspension. In a full process chain, according to various embodiments, the initial flocculation, and de-watering can lower the overall energy spent as well since the collected biomass can then be run through the system a second time at the lower flow rate, higher output, but with a concentrated (more dewatered) slurry to induce cell lysis and oil extraction.

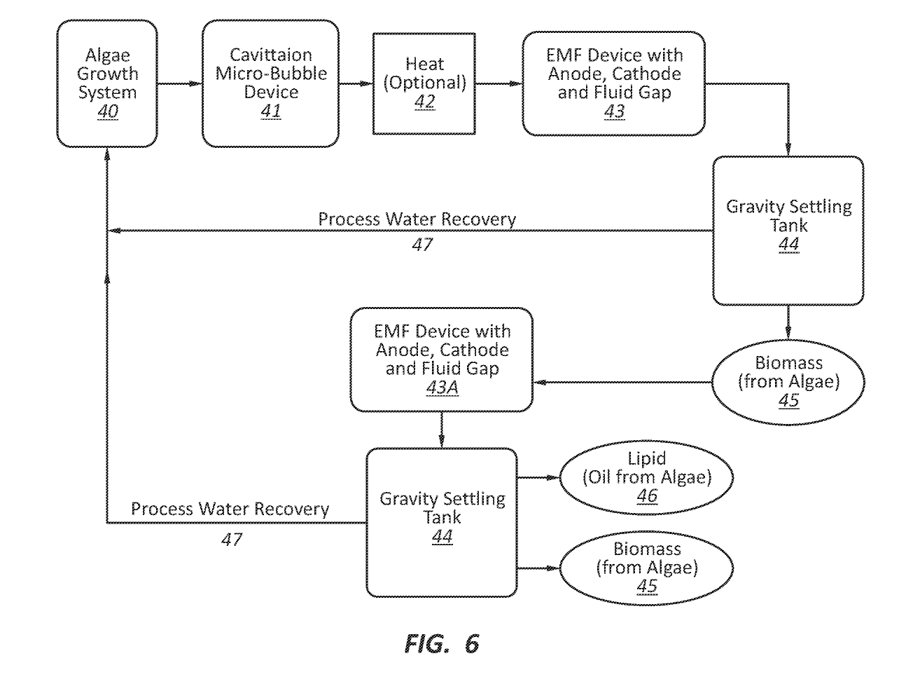

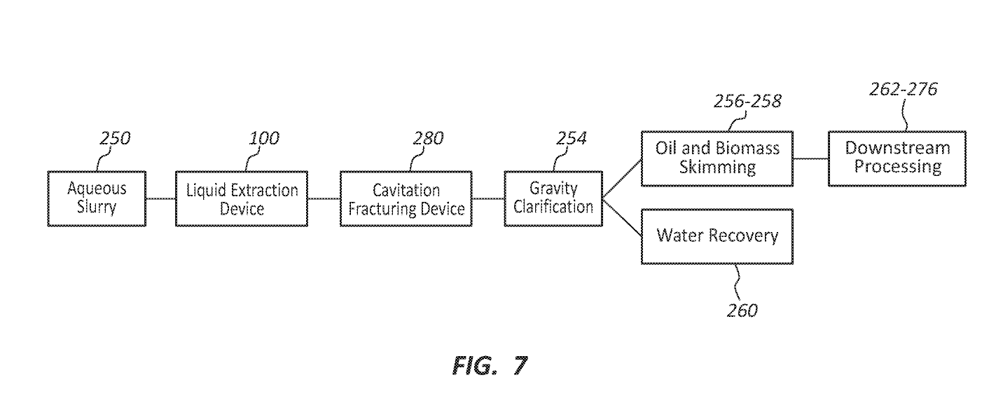

With reference now to FIG. 6 (see also FIGS. 7 & 11), a process according to some embodiments is illustrated. As shown in FIG. 6, in some embodiments, the use of electro-flocculation is used to obtain lipids from algae. For example, in step 40 algae, is grown to produce “green water”. According to some embodiments, the algae is grown in a growth chamber. In some embodiments, a growth chamber may comprise or be comprised of a reactor. In other embodiments, the algae may be grown in a natural or outdoor environment. For example, according to some embodiments, the growth chamber can be any body of water or container or vessel in which all requirements for sustaining life of the algae cells are provided. Examples of growth chambers include, but are not limited to, an open pond or an enclosed growth tank. When added to the flocculation device, the algae cells are generally in the form of a live slurry (also referred to herein as “biomass”) according to certain embodiments (see also FIGS. 7 & 11 at 250). In some embodiments, the live slurry is an aqueous suspension that includes algae cells, water and nutrients such as an algal culture formula based on Guillard's 1975 F/2 algae food formula (0.82% Iron, 0.034% Manganese, 0.002% Cobalt, 0.0037% Zinc, 0.0017% Copper, 0.0009% Molybdate, 9.33% Nitrogen, 2.0% Phosphate, 0.07% Vitamin B1, 0.0002% Vitamin B12, and 0.0002% Biotin) that provides nitrogen, vitamins and essential trace minerals for improved growth rates in freshwater and marine algae. In various embodiments, any suitable concentration of algae cells and sodium chloride, fresh, brackish or wastewater can be used, such that the algae cells grow in the aqueous suspension.

According to some embodiments, the algae may be harvested by drawing the aqueous algae slurry from the growth chamber using techniques known in the art. In at least one embodiment, the method of flocculating algae can be carried out by periodically drawing algae from a growth chamber in a manner that maintains a steady rate of growth. In such embodiments, steady state growth can be achieved by drawing algae at a rate of less than half the algae mass per unit time that it takes for the algae to double. In one embodiment, algae are harvested at least as often as the doubling time of the algae. In other embodiments, however, the algae are harvested at least twice during the doubling time of the algae. In various embodiments, the doubling time will depend on the algae type and growth conditions but can be as little as 6 hours to several days.

The method continues, according to some embodiments, through the use of cavitation. For example, according to such embodiments, prior to flocculation (step 43), the slurry can optionally be processed using cavitation (step 41, see also FIG. 7 at 280) and/or heating (step 42). As the method continues, the slurry is then flocculated using emf as described herein and according to various embodiments disclosed herein.

In some embodiments, the aqueous slurry is then delivered to the flocculating apparatus using any means, such as, but not limited to, gravity or a liquid pump. As the method continues according to various embodiments, the aqueous algae slurry is flowed via a conduit into an inlet section of an anode and cathode circuit of a flocculation device using any suitable device or apparatus, e.g., pipes, canals, or other conventional water moving apparatus(es). In some embodiments, a growth chamber or reactor is operably connected to the flocculating apparatus such that the algae cells within the growth chamber or reactor can be transferred to the apparatus as discussed above.

According to some embodiments, after the algae cells are flocculated in step 43, the flocculated slurry is dewatered (step 44). In such embodiments, dewatering (step 44) is carried out by separating a portion of the aqueous medium from the flocculated cells using any technique know in the art. In one embodiment, for example, the treated, flocculated cells can be harvested from the top of the tank such as by skimming or passing over a weir, where they can be recovered and/or further processed. In such embodiments, the flocculated cells can float to the surface by creating a bubble stream, either by cavitation of the creation of microbubbles from a microbubble generator, and impinging the bubbles beneath the flocculated mass of algae cells to cause them to rise to the surface in a froth. Further according to such embodiments, a skimming device is then used to harvest the froth floating on the surface of the liquid column. The remaining liquid (e.g., water) can be filtered and returned to the growth chamber (recycled) or removed from the system (discarded) according to various embodiments. In an alternative embodiment, the flocculated algae cells may be denser than the liquid medium and allowed to sink to the bottom of a settling tank (i.e., step 44). In such embodiments, the flocculated algae can be collected in a gravity settling tank (step 44, see also FIGS. 7 & 11 at 254) and the clarified water can be recycled (step 47). According to various embodiments, the dewatering step 44 produces a concentrated algae slurry 45. The concentrated aqueous algae slurry can then be used to extract lipids according to various embodiments (optionally using an extraction device that uses an emf) (step 43A).

Accordingly, various embodiments for dewatering and recovering microorganisms, such as algae cells, from an aqueous medium are disclosed with reference to FIG. 6. As discussed previously, such embodiments include subjecting algae cells in an aqueous suspension to an EMP in an apparatus as described herein, resulting in a magnetic effect or polar creation/enhancement of the algae cells and coagulation of the so treated algae cells from the aqueous medium. According to some embodiments, by the process of sinking in a settling tank, or the alternative process of causing the flocculated cells to float in a secondary tank, the algae cells can easily be removed from the aqueous medium. Thus, according to various embodiments, the algae cells in aqueous suspension can be coagulated, facilitating easy separation from the aqueous medium.

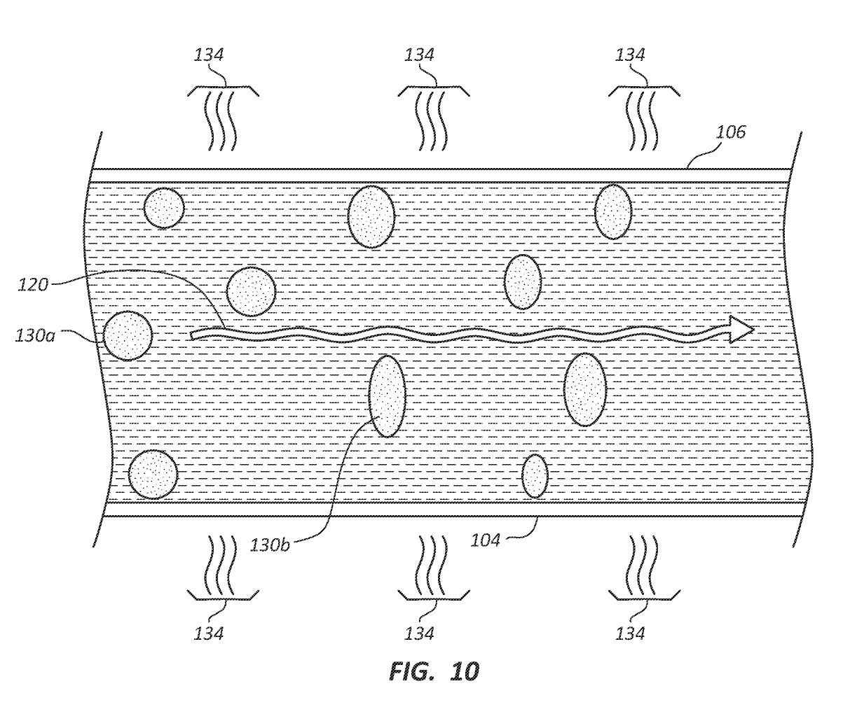

According to various embodiments, a method of harvesting cellular mass and debris from an aqueous solution containing algae cells (which in some embodiments may be referred to as “two step extraction”) includes subjecting algae cells to EMF and to either settling or floatation treatment (i.e., microbubbles) in an apparatus as described herein, resulting in a mixture that includes both coagulated cells and microbubbles or froth. In such embodiments, the cells can simply be skimmed off the surface of the aqueous medium by passing over a weir into a recovery tank as mentioned above. In at least one embodiment, the apparatus includes an anode, a cathode and a source of electric power, which is pulsed according to various embodiments and automatically controlled according to additional embodiments, to impart the necessary electromagnetic field to the cells. In such embodiments, the resulting coagulated cells are permitted to sink in a secondary, settling tank and separated from the aqueous medium. In another embodiment configured to facilitate dewatering cells from an aqueous medium containing algae cells, the algae cells in aqueous suspension can optionally be subjected to heat to achieve sinking of the coagulated cells within the settling tank, thereby facilitating separation of the coagulated cells from the aqueous medium. In various embodiments, heat can be applied to the culture containing the cells before (i.e., upstream of) the EMF, or heat can be applied to the culture in the apparatus (e.g., concomitantly with EMF as shown in FIG. 10).

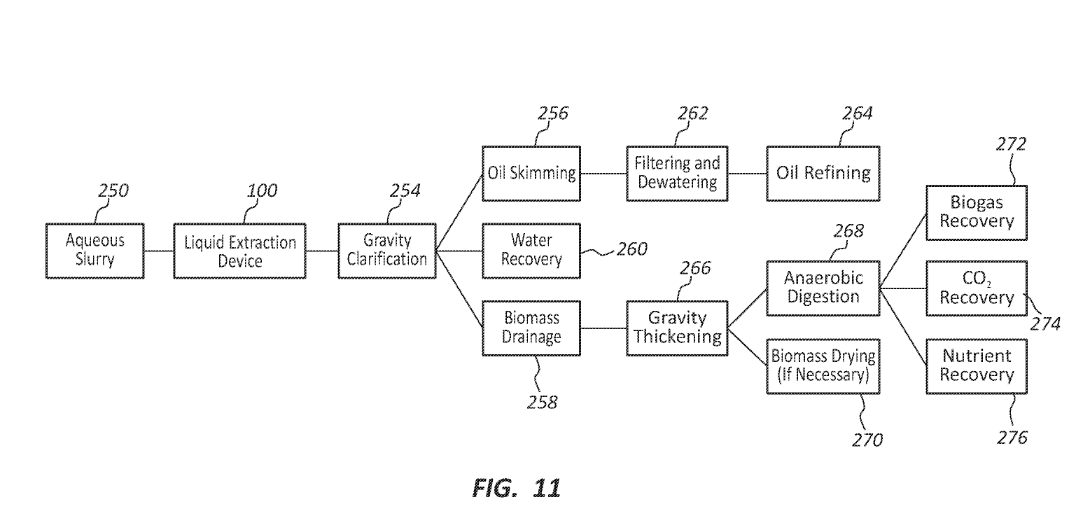

With continued reference to FIG. 6, and according to some embodiments, additional downstream processes may be carried out following flocculation and dewatering. According to such embodiments, example downstream treatments are numerous and may be employed depending on the desired output/use of the intracellular contents and/or bio cellular mass and debris mass. For example, lipids can be filtered by mechanical filters, centrifuge, or other separation devices. According to such embodiments, the lipids can then be heated to evacuate more water. According to further embodiments, the lipids can then be further subjected to a hexane distillation. In another example and/or other embodiments, cellular mass and debris is subjected to an anaerobic digester, a steam dryer, or belt press for additional drying for food, fertilizer etc. According to yet further embodiments, other downstream treatments also include, e.g., polishing and gravity thickening. For example, FIGS. 7 and 11 illustrates various downstream processes that can be performed on the dewatered algae and/or extracted lipids, such as, but not limited to, filtering and further dewatering 262, oil refining 264, gravity thickening 266, anaerobic digestion 268, biomass drying 270, biogas recover 272, CO₂ recovery 274 and/or nutrient recovery 276, among other downstream processes.

According to various embodiments, any suitable method may be used to extract the lipids. According to some embodiments, the cell membrane can be ruptured and the intracellular product recovered is oil (i.e., lipids). The lipids may also be processed, according to certain embodiments, into a wide range of products, including, but not limited to, vegetable oil, refined fuels (e.g., gasoline, diesel, jet fuel, heating oil, etc.), specialty chemicals, nutraceuticals, and pharmaceuticals, or biodiesel by the addition of alcohol. According to additional embodiments, the cellular mass and debris remaining after removal of the lipids, or other components of interest, e.g., proteins, can also be processed into a wide range of products, including biogas (e.g., methane, synthetic gas or syngas), liquid fuels (jet fuel, diesel), alcohols (e.g., ethanol, methanol), food, animal feed, and fertilizer, etc.

According to various embodiments, the flocculation process can be beneficial even if the desired outcome does not require the lysing of the cells, but just the biomass being separated from a portion of the water present from harvesting. According to some embodiments, for example, in a full process chain, the initial flocculation and de-watering can lower the overall energy spent as well since the collected biomass can then be processed in a lower volume. As mentioned above, according to some embodiments, the dewatered algae (i.e., concentrated algae slurry) can be used in any process for extracting lipids. However, as described more fully below, in one embodiment, the concentrated algae slurry is used in a lipid extraction process that uses emf to extract the lipids. In one embodiment, the same apparatus used for flocculation can be used for extraction by running the flocculated and dewatered slurry through the apparatus a second time at a lower flow rate, higher output, but with a concentrated (more dewatered) slurry to induce cell lysis and oil extraction. According to some embodiments, the concentrated slurry going through a second pass, or second EMF system downstream, will require (90%) less total biomass+water (as the biomass is now significantly dewatered) throughput to the second system thus effectively lowering the overall process energy requirement(s).

As mentioned above, in one embodiment, lipid extraction can be carried out using a lipid extraction device that releases lipids using emf (see FIGS. 7 & 11 at 100). In at least one embodiment, lipid extraction can be carried out using a lipid extraction device that releases lipids using pulsed emf. Examples of suitable apparatuses, methods, and systems for extracting lipids using emf are described in Applicants co-pending U.S. patent application Ser. No. 12/907,024, filed Oct. 18, 2010, and titled “Systems, Apparatuses, and Methods for extracting Non-Polar Lipids From an Aqueous Algae Slurry and Lipids Produced Therefrom,” the entire disclosure of which is hereby incorporated herein by reference.

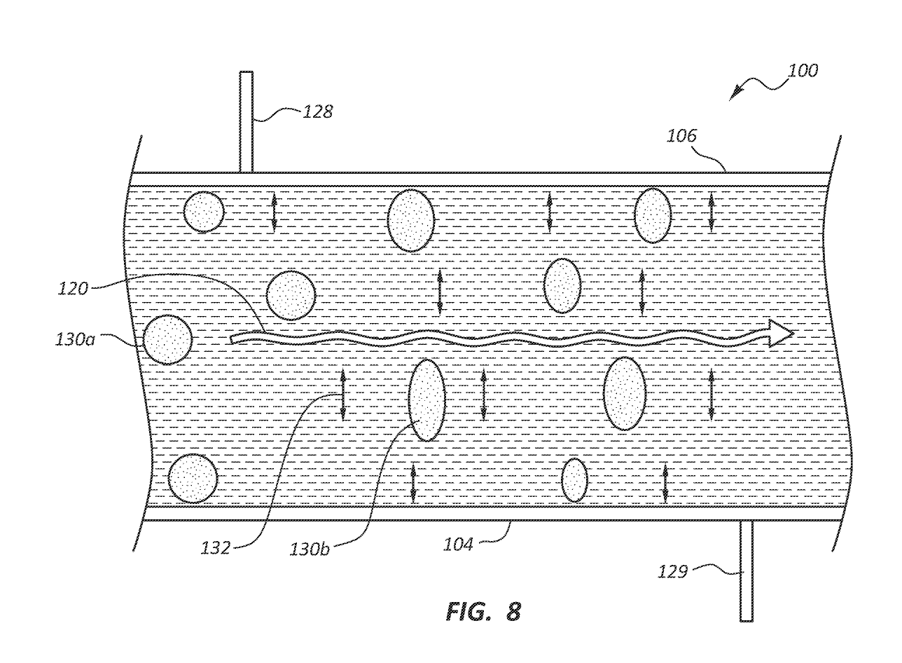

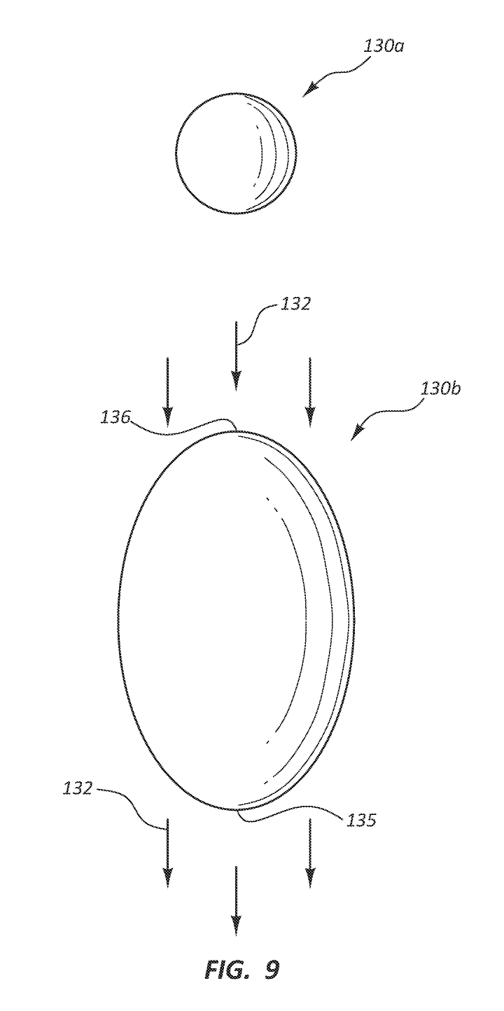

In general, according to various embodiments, the lipid extraction device can be constructed in a similar manner to the flocculation device described with respect to FIGS. 1-5 and discussed previously, except that the flow rate, gap distance, voltage and amperage are selected to create an emf that will compromise the algae cells and cause the intracellular lipids to be released. According to such embodiments, the release of lipids is caused by the stretching of the algae cells in the electric field to an extent that the algae cell wall and membrane are compromised or ruptured. Extraction by emf, according to such embodiments, is further explained with reference to FIGS. 8-9.

Turning now to FIG. 8, a simplified schematic is used to illustrate an emf transfer between two electrical conductive metal pieces with a liquid medium containing a living microorganism biomass flowing between them in an embodiment of a method for harvesting biomass from an aqueous solution containing algae cells. As shown in FIG. 8, according to some embodiments, cathode 106 requires a positive electrical connection point 128, which is used for positive current input according to some embodiments. According to such embodiments, positive transfer polarizes the entire length and width of cathode 106 and seeks a grounding source in anode 104. In order to complete an electrical circuit, according to such embodiments, anode 104 includes a grounding connection point 127, which allows an electrical transfer 132 to occur through aqueous slurry 120 according to some embodiments. In some embodiments, the aqueous slurry includes a liquid medium that contains a nutrient source mainly composed of a conductive mineral content that was used during a growth phase of the algae in aqueous slurry 120. According to such embodiments, the liquid medium containing the nutrient source further allows positive electrical input to transfer between cathode 106 through the liquid medium/biomass 120 to anode 104, which, according to some embodiments, only occurs when the liquid medium is present or flowing. In the foregoing embodiments, electrical input causes cellular elongation such as the distention shown in algae 130b as compared to algae 130a.

According to some embodiments, pulsing the electrical input phase contributes to cellular elongation 130b due to an electromagnetic field produced during an on cycle electrical phase. According to various embodiments, any suitable number, duration, for example, 60-80% duty cycle @ 1-2 kHz, of pulses can be used in connection with an appropriate wattage. According to various embodiments, as mentioned elsewhere, elongation of the cells is due to a positive and negative polarity response due to conductive minerals consumed as part of their nutrient uptake during growth and reproduction. In some embodiments, magnetic pulse response is useful in aiding in a creation/strengthening of the polar regions in the cell. According to some embodiments, once the pulsed electromagnetic field activates, the microorganism cells magnetically align with the most responsive positive side facing the anode 104 and with the negative responsive side facing the cathode 106. According to various embodiments, during the off cycle electrical phase the cells are allowed to relax. According to various embodiments, at a high frequency rate of electrical input, the cells are repeatedly stretched and relaxed similar to a thin piece of metal being flexed back and forward until the desired coagulation tendency is achieved. The foregoing analogy is similar to the experience encountered by the biomass cells during the on and off pulse phases which eventually aids in the lysis or fracturing process of the cell wall structure.

In reference to FIG. 9, a simplified illustration is used to exhibit the difference between a normal sized microalgae cell 130a in comparison to a microalgae cell 130b which has been extended by the electrical field between the cathode and anode pair according to various embodiments. As shown in FIG. 9, according to some embodiments, during the electrical “on” phase, emf 132 polarizes the algae cell walls and/or membranes. In such embodiments, a positive charge and a negative charge develop on the membrane of respective ends 135 and 136 of algae cell 130b in alignment with the emf field 132. According to such embodiments, the dipole on the cells causes the cells to be pulled apart along the electrical field lines until the cell wall/membrane is compromised, thereby releasing the cell contents or intracellular products. In such embodiments, this elongation eventually causes external structural damage to the exterior wall with general damage resulting in a wall and membrane that is permeable to the intracellular fluids and/or otherwise causes lysis. In various embodiments, the flow rate, voltage, and amperage, are selected in combination with the gap distance and composition of the aqueous slurry to cause release of primarily the polar lipids without releasing the non-polar lipids such as those in the cell membrane and the chlorophyll. According to various embodiments, a visual inspection or high performance liquid chromatography can be used to monitor the lipid content to minimize the polar lipid fraction as compared to the non-polar lipid fraction.

According to various embodiments, during the electrical on phase, pulsed electrical transfer 132 momentarily penetrates into intracellular components, which adsorb the energy transfer, resulting in momentary internal swelling to occur. According to such embodiments, this swelling produces pressure against the cell's wall structure due to internal component swelling beyond allotted space allowances. Conversely, according to such embodiments, during the off circuit phase, internal swelling decreases. However, according to various embodiments, repeated and/or cyclical on and off phases creates an internal pumping action as the contained internal mass swells and is forced up against the cell wall structure. According to such embodiments, this repeated pressure fluctuation combined with the electromagnetic field contributing to cellular pulsed elongation eventually causes external structural damage to the exterior wall with general damage resulting in inducement or strengthening of polar regions in the cells.

With continued reference to FIG. 9, in some embodiments, an electrical pulse is repeated in frequency to create an electromagnetic field and electrical energy transfer between two electrically conductive materials, such as metal pieces according to some embodiments, when a conductive liquid medium containing a living microorganism biomass is flowed between them. As this pulsed electrical transfer occurs, an electromagnetic field is produced resulting in the elongation of the biomass cells due to their polarity according to certain embodiments. According to further embodiments, the suspended biomass absorbs electrical input which causes internal cellular components and their liquid mass to swell in size. In such embodiments, and due to swelling, an internal pressure is applied against the transmembrane, however this internal swelling is to be considered as only momentary according to certain embodiments as it is relieved during an off frequency phase of the pulsed electrical input. As mentioned above, in some embodiments, rapid repeating of the on and off electrical frequency rearranges components and creates and/or increases the polar regions in the algae cells. In some embodiments, continuous frequency inputs further produce internal pressures caused by expanded internal component swelling which eventually creates the magnetic/electrostatic attraction causing coagulation/flocculation of the treated cells.

Additionally, in some embodiments, the amount of electrical voltage and/or current or frequency input can be adjusted based on a matrix formula of grams of biomass contained in 1 liter of the liquid medium.

For extraction, in one embodiment, the flow rate through the gap volume (i.e., the portion of the channel in the electric field at the gap distance) is 0.1 ml/second per ml of gap volume. In other embodiments, the flow rate through the gap volume is at least 0.5 ml/second per ml of gap volume. In still other embodiments, the flow rate through the gap volume is at least 1.0 ml/second per ml of gap volume. And, in still other embodiments, the flow rate through the gap volume is at least 1.5 ml/second per ml of gap volume. In various embodiments, the flow rate through the gap volume exceeds 1.5 ml/second per ml of gap volume. In at least one embodiment, the flow rate can be controlled by controlling the pressure using a pump or other suitable fluid flow mechanical device(s).

According to various embodiments, the average amperage is at least 1 amp, 5 amps, 10 amps, 50 amps, or even at least 100 amps. In other embodiments, however, the maximum amps can be less than 200 amps, less than 100 amps, less than 50 amps, or less than 10 amps. The range of amperage can be any range from the foregoing maximum and minimum amperages according to various embodiments.

Likewise, in some embodiments, the voltage can be at least 1V, 10V, 100V, 1 kV, or even at least 20 kV. In other embodiments, the maximum voltage can be less than 50 kV, less than 30 kV, less than 10 kV, less than 1 kV, or less than 100V. The range of voltage can be any range of the foregoing maximum and minimum voltages according to various embodiments.

An example of a suitable configuration for extracting non-polar lipids according to some embodiments includes an apparatus with a gap distance of 1/16-¼ inch and a gap volume of 250 ml-1000 ml and an electrical current of 1-60 peak amps @1-24 volts or 25w to 500 watts. The flow volume, in such embodiments, can be at a rate of 1 gallon per minute (GPM) of throughput with a culture having a density of 500 mg/L. In such embodiments, one would use approximately 70 watts of energy (3.5v @20 peak amps) for a successful extraction. At 5 GPM, the same culture could be extracted using approximately 350 watts (3.5v @100 peak amps) according to alternative embodiments.

In another example, according to alternative embodiments, at 0.5 GPM and 500 mg/L density, an electrical current of approximately 60 watts (15 peak amps @4 volts) is applied. In some embodiments, a GPM of approximately 0.1 to approximately 5 GPM and watts in the range of about 20 to about 1000 watts (e.g., 2-18 volts @2-50 peak amps) are used. For example, in some embodiments, at 1 GPM of throughput with a culture having a density of 500 mg/L, one could use approximately 70 watts of energy (3.5v @20 peak amps) for a successful extraction. At 5 GPM, the same culture would require approximately 350 watts (3.5v @100 peak amps) according to alternative embodiments.

In at least one embodiment, an emf can be pulsed on and off repeatedly to cause cyclical extension(s) and relaxation(s) of the algae cells. In this embodiment, voltages can be higher and peak amperage lower while average amperage remains relatively low. In such embodiments, this reduces the energy requirements for operating the apparatus and reduces wear on the anode and cathode pair. In one embodiment, the frequency of the emf pulses is at least about 500 Hz, 1 kHz, 2 kHz, or 30 kHz. In various embodiments, the frequency can be less than 200 kHz, 80, kHz, 50 kHz, 30 kHz, 5 kHz, or 2 kHz. Ranges for the pulse frequency can be any combination of the foregoing maximum and minimum frequencies according to various embodiments.

According to some embodiments, as discussed in general previously, the temperature of the aqueous slurry during extraction can also have an impact on the power required to extract the non-polar lipids. According to some embodiments, for example, lipid extraction is carried out at room temperature. However, in other embodiments, heat is added to the aqueous algae slurry to achieve a desired or alternate temperature. For example, in some embodiments, lipid extraction may be carried out at a temperature above 40° F., 65° F., 80° F., 100° F., or 120° F. In other embodiments, however, the temperature is below 130° F., 115° F., 105° F., or 90° F. Ranges for the extraction temperature can be any combination of the foregoing maximum and minimum temperatures according to the various embodiments disclosed herein.

In some embodiments, the temperature of the slurry can also be adjusted to control the specific gravity of the water relative to the algae (the specific gravity of water density is optimal at 40 degrees F. according to the various embodiments). In such embodiments, as the liquid medium (typically composed primarily of water) is heated, alterations to its hydrogen density occurs; this alteration of density allows a normally less dense material to sink or in this case, heavier fractured cellular mass and debris materials which would normally float, now rapidly sink to the bottom of the liquid column. This alteration, according to some embodiments, also allows easier harvesting of these materials, which are also useful for other product applications according to various embodiments. Once the EMP and heating process has been achieved, according to the foregoing embodiments, the liquid medium containing a now fractured biomass is transferred into a secondary holding tank where a liquid pump allows a continuous loop flow with respect to various embodiments. As used in this description “specific gravity” is a dimensionless unit defined as the ratio of density to a specific material as opposed to the density of the water at a specified temperature.

In reference to FIG. 10, a simplified schematic is used to illustrate a heat transfer, according to some embodiments, between the outer walls of the cathode 106 and/or anode 104 and into the liquid medium/biomass during the EMP process in a method for harvesting cellular mass and debris from an aqueous solution containing algae cells. In such embodiments, an applied heating device 134 attaches to the outside wall surfaces of cathode 106 and anode 104, which allows heat transfer to penetrate into the aqueous slurry 120. According to various embodiments, changes to the specific gravity of the liquid medium, which is mainly composed of water, by heating allows alteration in its compound structures which is mainly due to alterations to the hydrogen element which when altered, lessens the water density. In such embodiments, this density change allows a normally less dense material contained within a water column to sink or, in some embodiments, a coagulated mass of algae cells to sink.

According to certain embodiments, the products recovered from the methods of the present invention can have a relatively low content of polar lipids, such as chlorophyll and phospholipids. In at least one embodiment, lipid extraction is carried out to produce a released lipid fraction with a non-polar lipid content greater than 80% and the polar fraction is less than 20%. In other embodiments, the non-polar lipid content in the released fraction is greater than 90% and the polar content is less than 10%. In yet additional embodiments, the non-polar lipid content is greater than 95% and the polar lipid content is less than 5%. In still other embodiments, the non-polar lipid content is greater than 98% and the polar lipid content is less than 2%. In yet another embodiment, the non-polar lipid content is at least 99% and the polar lipid content is less than 1%.

According to various embodiments, various methods may further include reducing the content of phosphorus to less than 100 ppm. In other embodiments, various methods further reduce the content of phosphorus to less than 20 ppm. In still other embodiments, various methods further reduce the content of phosphorus to less than 10 ppm. In some embodiments, either the polar lipids or the non-polar lipids are employed in at least one catalytic refining process. For example, the lipids can be hydrotreated using a supported catalyst.

In one embodiment, the present invention includes a method of harvesting cellular mass and debris from an aqueous solution containing algae cells by subjecting algae cells to pulsed emf and to cavitation (i.e., microbubbles) in an apparatus as described herein, resulting in a mixture that includes both intracellular product(s) (e.g., lipids) and cellular mass and debris. A process flow diagram that includes a cavitation step according to such embodiments is illustrated in FIG. 11 (see also FIG. 6 at 41). The methods and apparatus of this embodiment can use any of the lipid extraction devices described herein. According to such embodiments, the algae cells are subjected to cavitation before application of (upstream of) pulsed emf (i.e., “EMP”), or they may be subjected to cavitation concomitantly with the application of an EMP. In one embodiment, a cavitation device includes an anode, a cathode and venturi mixer (not shown) (all in one). In such embodiments, the cavitation unit is reduced (e.g., by half), a non-conductive gasket is added, and it is electrified. According to such embodiments, and under normal pressure conditions, e.g., under 100 psi, the application of cavitation upstream of EMP has no effect. However, according to various embodiments, at pressures above 100 psi (e.g., 110, 115, 120, 130, 140, 150, 200, 300, 400 psi, etc.), the application of cavitation has an effect on the processes disclosed herein. It should be noted that, as contemplated herein, any venturi device as commonly understood by those of skill in the art may be employed to carry out the various methods disclosed herein.

According to various embodiments, after the non-polar lipid fraction is released, the released lipid fraction may be subjected to one or more downstream treatments including gravity clarification 254, as shown in FIG. 11. According to some embodiments, gravity clarification generally occurs in a clarification tank in which the coagulated algae mass sinks to the bottom of the tank. In such embodiments, upon transiting the circuit, the electromagnetically treated algae cells are flowed into a gravity clarification tank that is operably connected to a flocculation apparatus for treating algae cells as described herein. In the gravity clarification tank, according to various embodiments, the treated, individual cells, coagulated with similarly treated cells, form a coagulated mass which sinks to the bottom for harvest.

Various additional downstream process take place subsequently thereto according to various embodiments, such as, but not limited to, oil skimming 256, water recovery 260, biomass draining 258, filtering and further dewatering 262, oil refining 264, gravity thickening 266, anaerobic digestion 268, biomass drying 270, biogas recover 272, CO₂ recovery 274 and/or nutrient recovery 276, among other downstream processes.

According to embodiments wherein cavitation is used, a micron mixing device, such as a static mixer or other suitable device such as a high throughput stirrer, blade mixer or other mixing device is used to produce a foam layer composed of microbubbles within a liquid medium containing a previously lysed microorganism biomass. According to such embodiments, any device suitable for generating microbubbles, however, can be used. In such embodiments, following micronization, the homogenized mixture begins to rise and float upwards. According to such embodiments, as this mixture passes upwards through the liquid column, the less dense valuable intracellular substances freely attach to the rising bubbles, or due to bubble collision, rise to the surface. Further according to such embodiments, the heavier sinking cellular mass and debris waste is then allowed to sink according to heated water specifics. The rising bubbles also shake loose trapped valued substances (e.g., lipids) which also freely adhere to the rising bubble column according to the foregoing embodiments.

Once the foam layer containing these useful substances has risen to the top of the liquid column as described with reference to some embodiments, the valuable intracellular substances or products now can be easily skimmed from the surface of the liquid medium and deposited into a harvest tank for later product refinement or other subsequent processes according to various embodiments. According to some embodiments, once the foam layer rises to the top of the secondary tank, the water content trapped within the foam layer generally results in less than 10% (e.g., 5, 6, 7, 8, 9, 10, 10.5, 11%) of the original liquid mass. Trapped within the foam, according to such embodiments, are the less dense useful substances, and the foam is easily floated or skimmed off the surface of the liquid medium. According to various embodiments, this process requires only dewatering of the foam, rather than evaporating the total liquid volume needed for conventional harvest purposes. Such embodiments drastically reduce the dewatering process, energy and/or any chemical inputs while increasing harvest yield and efficiency as well as purity. Further, in such embodiments, water can be recycled to the growth chamber or removed from the system. Likewise, cellular mass and debris can be harvested at any appropriate time, including, for example, daily (batch harvesting) according to various embodiments. In another example or alternative embodiments, cellular mass and debris is harvested continuously (e.g., a slow, constant harvest).

According to various embodiments, once the liquid medium has achieved passage through the EMP apparatus, it is allowed to flow over into a secondary tank (or directly into a device that is located near the bottom of the tank). In such embodiments adapted for dewatering, the secondary tank is a tank containing a micron bubble device or having a micron bubble device attached for desired intracellular component separation and dewatering. According to such embodiments, after transmembrane lysis, a static mixer or other suitable device (e.g., any static mixer or device which accomplishes a similar effect of producing microbubbles) is used and is located at the lowest point within a secondary tank. When activated, the static mixer produces a series of micron bubbles resulting in a foam layer that develops within the liquid medium. As the liquid medium is continuously pumped through the micro mixer, according to various embodiments, bubbled foam layers radiate outwards through the liquid and begin to rise and float upwards. In such embodiments, the less dense desired intracellular components suspended within the liquid medium attach to the micron bubbles floating upwards and flocculate to the surface or are detached from heavier sinking biomass waste (which is allowed to sink due to specific gravity alterations) due to rising bubble collisions within the water column.

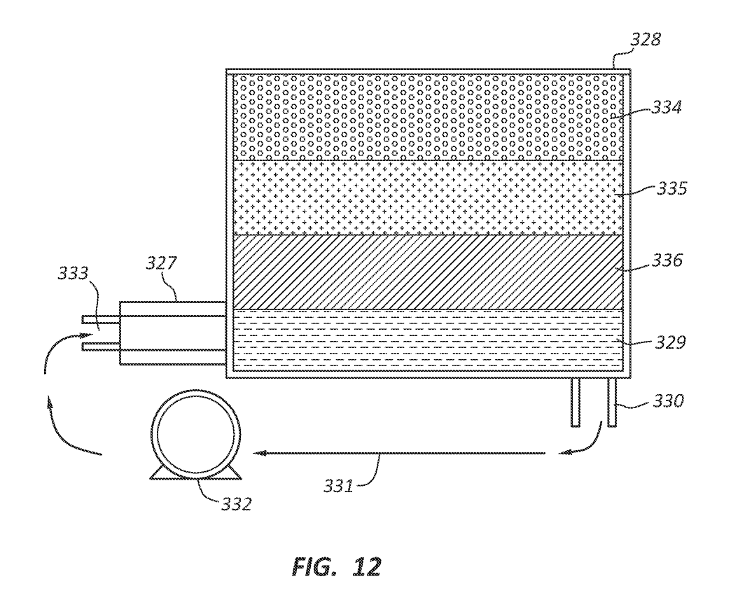

According to various embodiments, FIG. 12 illustrates a lower mounting location for a micron mixer 327 when in association with secondary tank 328 and containing a previously fractured biomass 329 suspended within a liquid medium. In such embodiments, the liquid medium is then allowed to flow through a lower secondary tank outlet 330 where it is directed to flow through conduit 331 having a directional flow relationship with a liquid pump 332. Further according to such embodiments, and due to pumping action, the liquid is allowed a single pass through, or to re-circulate through the micron mixer via a micron mixer inlet opening 333. In embodiments wherein the liquid continues to cyclically flow through micron mixer 327, microscopic bubbles 334 are increasingly produced relative to each cycle. In such embodiments, micro bubbles 334 radiate outwards within the liquid column 335, forming a foam layer 336. As the process continues according to certain embodiments, the composed layer starts to rise upwards towards the surface of the liquid column 335. In some embodiments, once the foam layer 336 starts its upward journey towards the surface of the liquid column 335, pump 332 is shut down, and thus the micronization process is complete. According to such embodiments, this allows all micron bubbles 334 produced at the lower exit point of the micron mixer 327 to rise to the surface, and, as they do, they start collecting valuable intracellular substances released into the liquid medium during the EMP process previously described. Further according to such embodiments, this upward motion of the micron bubbles 334 also rubs or bumps into heavier downward-sinking cellular mass and debris, further allowing the release of trapped lighter valuable substances that have bonded with or are otherwise trapped by heavier-sinking cellular mass and debris remains.

In various embodiments, once detached, these substances adhere to the micron bubbles 334 floating upwards towards the surface. In some embodiments, pump 332 remains on and continues to produce additional micron bubbles even after the foam layer 336 starts its upward journey. According to such embodiments, the system depicted in FIG. 12 is allowed to continually process an ongoing flow being introduced to the secondary tank 328.

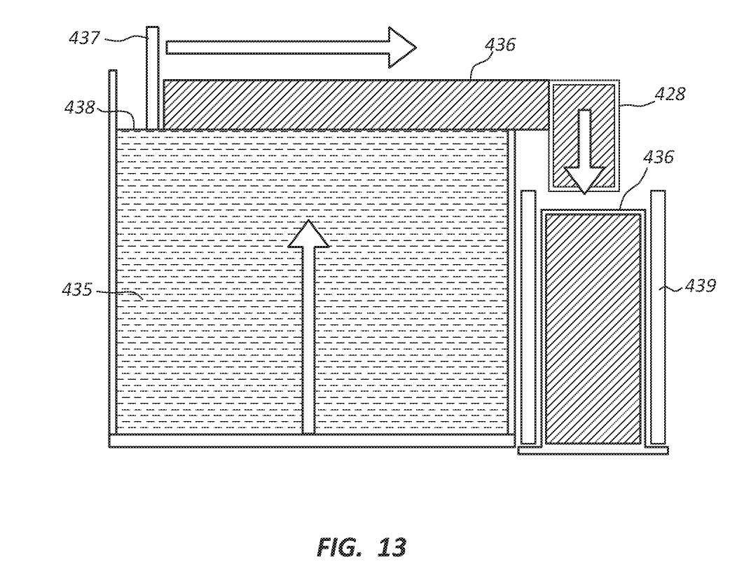

In reference to FIG. 13, a simple illustration is used to show a method according to various embodiments for harvesting a foam layer 436 containing approximately ten percent (10%) of the original liquid medium mass/biomass 435. According to such embodiments, as the foam layer 436 containing the valuable intracellular internal substances rises to the surface of the liquid medium 435, a skimming device 437 can be used to remove the foam layer 436 from the surface 438 of liquid medium 435. In various embodiments, the skimming device 437 located at the surface area of the secondary tank 428 allows the foam layer 436 to be pushed over the side wall of the secondary tank 428 and into a harvesting container 439 where the foam layer 436 is allowed to accumulate for further substance harvesting procedures. The apparatuses described with respect to FIGS. 12 & 13 may also be useful for carrying out the dewatering step of the present invention as described above with respect to flocculation and dewatering according to various embodiments.

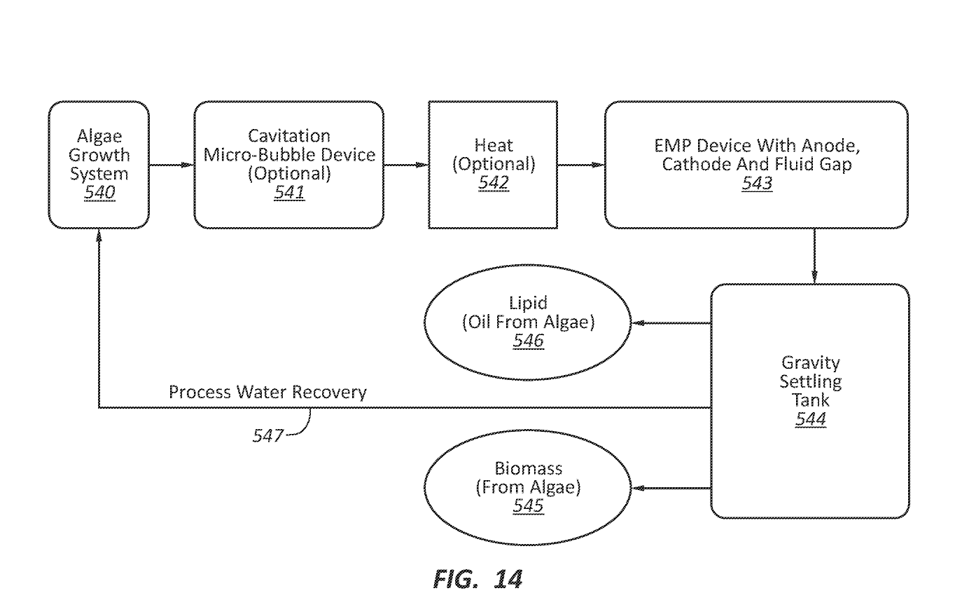

FIG. 14 illustrates one embodiment of a method and apparatus (system) as described herein for the harvest of useful substances from an algae biomass. As shown in FIG. 14, and according to various embodiments, microorganism algae are grown in a containment system 540 and, at the end of an appropriate growth cycle, are transferred into the substance recovery process discussed in detail above. In some embodiments, the algae biomass is flowed through an optional micron bubble cavitation step 541 (as discussed above), wherein the cavitation step 541 is used to soften the outer cellular wall structure prior to other bio substance recovery processes according to various embodiments.

According to further embodiments, after cavitation step 541, an optional heat process 542 can be applied to change the gravity specifics of the liquid feed stock water containing the biomass. In some embodiments, the heat option 542 allows a faster transfer of particular substances released during the harvest process. According to some embodiments, after the biomass has reached an appropriate heat range, it is then allowed to flow through an electromagnetic pulse field, the EMP station 543, where transiting biomass cells are exposed to electromagnetic transfers resulting in either the flocculation and dewatering of the cells or the fracturing and lysing of the outer cellular wall structures depending on various factors, such as voltage, residence time, pulse frequency and so forth as discussed above.

In various embodiments, once the algae slurry is flowed through the EMP process 543, the fractured biomass transitions into a gravity clarifier tank 544 where heavier matter (ruptured cell debris/mass) 545 sinks down through the water column as the lighter matter (intracellular products) 546 rises to the surface where it is harvested. In some embodiments, the heavier sinking mass 545 gathers at the bottom of the clarifier tank 544 where it can be easily harvested for other useful substances. According to some embodiments, after substance separation and recovery, the remainder of the water column 547 is sent through a water reclaiming process and after processing is returned back into the growth containment system 540 such that it can be used to facilitate the process described above on a cyclical basis.

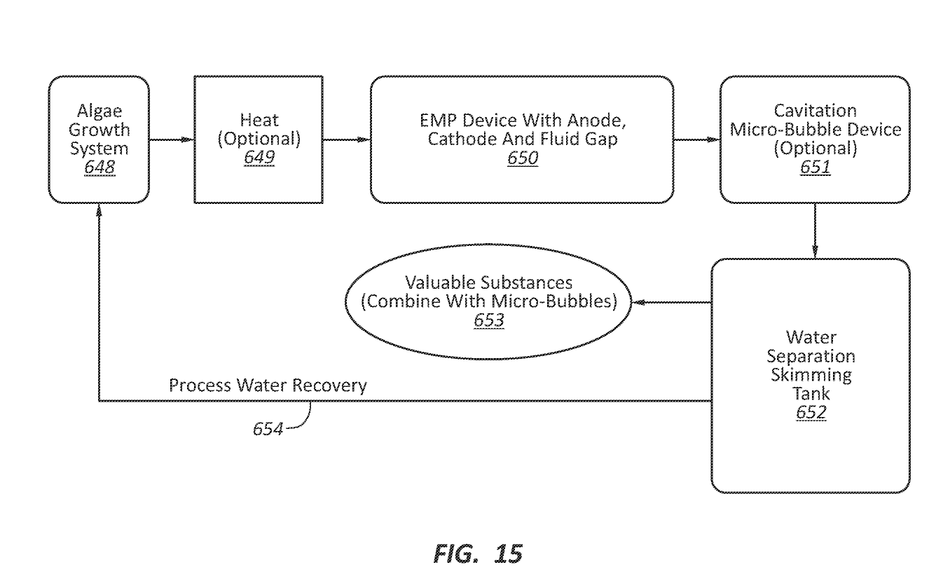

FIG. 15 illustrates another embodiment of a method and apparatus (system) as described herein for the harvest of useful substances from an algae biomass. According to various embodiments, microorganism algae are grown in a containment system 648 and, at the end of an appropriate growth cycle, are then transferred into the substance recovery process described in detail above. In some embodiments, the substance recovery consists of the algae biomass being transferred into an optional heat process 649 where the biomass water column is subjected to heat prior to entering the EMP station 650 or otherwise being subjected to an EMP. In other embodiments, no heat process is applied; rather, the slurry is simply processed at the ambient temperature. According to certain embodiments, after the EMP process (described in detail above), the fractured biomass is then transferred over into a cavitation station 651 where micron bubbles are introduced at a low point in a water column containment tank 652. In such embodiments, as the micro-bubbles rise through the water column, the valuable released bio substances (intracellular products) 653 attach to the rising bubbles which float to the surface of the water column allowing an easier and faster skimming process for substance recovery. In various embodiments, after substance recovery, the remainder of the water column is sent through a water reclaiming process 654 and after processing is returned back into the growth system 648 for subsequent reuse.

### ILLUSTRATIVE EXAMPLES

### Example 1

According to at least one embodiment, an algae culture comprising Nannochloropsis oculata (at an algae concentration of 200 mg/L dry weight) and pH 8.6 is passed through a stainless steel tube equipped with rifling and a gap of 0.125 inches. In such embodiments, a voltage of 12V, amperage of 30 amps pulsed at 1 kHz is applied across the gap. Further according to such embodiments, ambient temperature is 80° F., the culture being heated to 107° F. immediately before being introduced into the EMF inducing tube. In such embodiments, the culture is passed through the tube at a flow rate of 2 gallons per minute. According to the embodiment of Example 1, after passing through the EMF system, the treated sample is sent to a 3 Liter, 3″ diameter, clear PVC vertical clarifier and timed from the start as the fluid enters the EMF. In such embodiments, there is a relatively fast (10 minutes) flocculation in which the biomass rises to the surface of the clarifier. In such embodiments, there is no sign of cell lysis (microscope, 40× magnification).

### Example 2

According to a further embodiment, the procedure of Example 1 is repeated except that the amperage is decreased to 25 amps. In this embodiments, the algae flocculates and rises to the surface in about 15 minutes, then aggregates over an additional 25 minutes and sinks to the bottom of the clarifier. In this embodiment, there is no sign of cell lysis (microscope, 40× magnification).

### Example 3

According to another embodiment, the procedure of Example 2 is repeated except that the amperage is decreased to 20 amps. In such embodiments, the algae flocculates and rises to the surface in 25 minutes, then aggregates over an additional 21 minutes and sinks to the bottom of the clarifier. In this embodiment, there is no sign of cell lysis (microscope, 40× magnification).

### Example 4

In yet another embodiment, the procedure of Example 1 is repeated except testing is conducted at the ambient temperature of 76.6 degrees F. and the culture is not heated. In such embodiments, the algae culture sample Nannochloropsis oculata has a concentration of 200 mg/L dry weight, a pH of 8.6 and is passed through a stainless steel tube equipped with rifling and a gap of 0.125 inches, at a voltage of 12V, amperage of 30 amps and 1 KHz. In the embodiment of Example 4, the culture is passed through the tube at a flow rate of 2 gallons per minute (GPM). In such embodiments, there is a more modest rate of flocculation (35 minutes) in which the biomass rises to the surface of the clarifier. In this embodiment, there is no sign of cell lysis, (microscope, 40× magnification).

### Example 5

In yet another embodiment, the procedure of Example 2 is repeated except at ambient temperature of 76.8 degrees F. and the culture is not heated. In such embodiments, the algae flocculates and rises to the surface after 46 minutes, then aggregates over an additional 32 minutes and sinks to the bottle of the clarifier. In this embodiment, there was no sign of cell lysis (microscope, 40× magnification).

### COMPARATIVE ILLUSTRATIVE EXAMPLES

### Example 6

### Comparative

In still another embodiment, the procedure of Example 1 is repeated except that the amperage was increased to 35 amps and the flow rate through the tube was reduced to 0.75 GPM. In this embodiment, the cells are visually compromised and disfigured (microscope 40×) indicating cell lysis. In such embodiments, the biomass does not float but rather sinks fairly rapidly (10 minutes) and an oily film floats on the top of the aqueous system.

### Example 7

### Comparative

In yet another embodiment, the procedure of Example 1 is repeated except that the amperage is increased to 35 amps and the flow rate through the tube is reduced to 0.75 GPM and testing is conducted at ambient temperature of 76.9° F. In this embodiment, the cells are visually compromised and disfigured (microscope 40× magnification) indicating cell lysis.

The present invention may be embodied in other specific forms without departing from its spirit or essential characteristics. The described embodiments are to be considered in all respects only as illustrative and not restrictive. The scope of the invention is, therefore, indicated by the appended claims, rather than by the foregoing description. All changes which come within the meaning and range of equivalency of the claims are to be embraced within their scope.

What is claimed and desired to be secured by Letters Patent is:

1. A method for extracting and recovering lipids from microalgae, comprising:
providing an aqueous slurry of microorganisms, wherein at least 70 wt % of the microorganisms are microalgae cells;
extracting at least a portion of intracellular lipids from the microalgae cells;
recovering at least a portion of the intracellular lipids extracted from the microalgae cells.

2. The method of claim 1, further comprising the step of flocculating the microalgae cells prior to extracting the intracellular lipids from the microalgae cells.

3. The method of claim 2, wherein the step of flocculating the microalgae cells comprises applying a first electromotive force to the aqueous slurry.

4. The method of claim 3, wherein the aqueous slurry has at least 2% electro-flocculated microalgae by weight of the aqueous slurry.

5. The method of claim 3, further comprising the step of removing a portion of water from the aqueous slurry to form a concentrated microalgae slurry prior to extracting the intracellular lipids from the microalgae cells.

6. The method of claim 5, wherein extracting at least a portion of the intracellular lipids is carried out by applying a second electromotive force to the microalgae in the concentrated microalgae slurry.

7. The method of claim 6, wherein applying a second electromotive force to the microalgae is carried out by flowing the concentrated microalgae slurry between two electrodes that, at least in part, form a channel that defines a fluid flow path in order to apply the second electromotive force to the concentrated microalgae slurry such that the microalgae cells are compromised and therefore release the intracellular lipids.

8. The method of claim 7, wherein the two electrodes are separated by a gap with a distance in a range from 0.5 mm to 200 mm.

9. The method of claim 8, wherein the second electromotive force is applied across the gap.

10. The method of claim 9, wherein the aqueous slurry is caused to flow through the gap at a rate of at least 1.0 ml per second per ml of gap volume.

11. The method of claim 10, wherein the channel defines a spiral fluid flow path.

12. The method of claim 11, wherein one of the first electromotive force and the second electromotive force are selectively pulsed.

13. The method of claim 12, wherein one of the first electromotive force and the second electromotive force is pulsed at a frequency of at least 1 kHz.

14. The method of claim 13, wherein an amperage used to create one of the first electromotive force and the second electromotive force is at least 1 amp.

15. The method of claim 14, wherein a voltage used to create one of the first electromotive force and the second electromotive force is at least 1V.

16. The method of claim 15, wherein the gap volume of the fluid flow path is at least 200 ml.

17. The method of claim 1, wherein the intracellular lipids are extracted, at least in part, by one of centrifuging, drying and milling.

18. The method of claim 1, further comprising the step of allowing cellular mass and debris to sink to the bottom of a container following extraction and recovery of the intracellular lipids.

19. The method of claim 1, further comprising the step of skimming the intracellular lipids from the surface of the aqueous medium following extraction.

20. The method of claim 1, further comprising the step of introducing microbubbles into the aqueous slurry following extraction in order to cause the intracellular lipids to rise to the surface of the aqueous slurry

21. An apparatus for dewatering algae cells from algae cells in aqueous suspension, the apparatus comprising:
at least one first electrical conductor that acts as a cathode and a second electrically conductive housing that acts as an anode, the at least one first conductor being disposed within the housing, such that a space is defined between the exterior of the first conductor and an interior of the housing, providing a flow path for the aqueous suspension;
an electrical power source operably connected to the first conductor and the housing for creating a first electromagnetic field (EMF) by providing an electrical current that is applied between the first conductor and the housing and the aqueous suspension; and
a separation tank in fluid communication and downstream of the first electrical conductor and the housing, the separating tank collecting flow from the first electrical conductor and the housing.

22. The apparatus of claim 21, wherein the first conductor and second housing are tubes.

23. The apparatus of claim 21, wherein the fluid flow is a spiral fluid flow.

24. The apparatus of claim 21, wherein the first conductor and second housing are each metal tubes.

25. The apparatus of claim 24, wherein the first conductor and second housing are metal tubes of circular shape.

26. The apparatus of claim 24, wherein the metal tubes are of different shapes.

27. The apparatus of claim 21, wherein the inner diameter of the metal housing and the outer diameter of the first conductor are in a range from 0.5 mm to 100 mm to a point of non-conductive transfer of electric current.

28. The apparatus of claim 21, wherein the housing is a metal tube and the at least one electrical conductor comprises a plurality of spaced apart electrical conductors, the electrical conductors being separated from each other by electrically insulating elements; and a multiplicity of flow paths being created between the housing and each of the plurality of spaced apart electrical conductors.

29. The apparatus of claim 28, wherein each of the plurality of electrical conductors are metal tubes.

30. The apparatus of claim 21, wherein the algae cells are induced to coagulate due to the application of the first electromagnetic field to the aqueous suspension.

31. The apparatus of claim 30, wherein the separation tank is a settling tank and the coagulated algae cells sink to the bottom of the tank.

32. The apparatus of claim 30, further comprising means for creating a source of bubbles in the lowermost portion of the tank to lift the coagulated algae cells to the upper surface of the aqueous suspension.

33. The apparatus of claim 32, further comprising an element to skim the coagulated algae cells from the separation tank.

34. The apparatus of claim 21, wherein the electrical power source provides a pulsed electrical current.

35. The apparatus of claim 21, further comprising a second electromagnetic field (EMF) through which the algae cells treated by the first EMF must pass through.

36. A method of dewatering algae cells from an aqueous suspension containing algae cells comprising the steps of:
providing the apparatus of claim 21, the apparatus further comprising an aqueous suspension comprising conductive minerals and algae cells wherein the aqueous suspension is disposed in the flow path of the apparatus;
applying a sufficient amount of an electrical current to the at least one first conductor and the housing and aqueous suspension for aligning the cells tending to cause the algae cells to coagulate with similarly treated algae cells in the aqueous suspension;
flowing the aqueous suspension containing the mass of treated algae cells into a separation tank and coagulating the algae cells in the separation tank; and,
separating the coagulated algae cells from the aqueous suspension in said separation tank.

37. The method of claim 35, further comprising the step of permitting the coagulated algae cells to sink to the bottom of the tank.

38. The method of claim 35, further comprising introducing bubbles into the bottom of the secondary tank causing the coagulated algae cells to rise to the surface of the aqueous medium.

39. The method of claim 37, further comprising the step of skimming the coagulated algae cells from the surface of the aqueous medium.

40. The method of claim 33, further comprising providing the electrical current as a pulsed current.

41. A method of dewatering algae cells and, in a second step, disrupting the algae cells' membrane releasing at least oil therefrom within an aqueous suspension comprising the steps of:
providing an aqueous suspension comprising conductive minerals and algae cells wherein the aqueous suspension is disposed in the flow path of a first electromagnetic field (EMF);
applying a sufficient amount of an electrical current to at least one first conductor and a housing through which the aqueous suspension is flowing at conditions for aligning the cells tending to cause the algae cells to coagulate with similarly treated algae cells in the aqueous suspension;
flowing the aqueous suspension containing the mass of treated algae cells through a second EMF under conditions such that the cell membranes of the coagulated algae cells are disrupted thereby releasing at least oil therefrom; and
flowing the algae cells and oil into a separation tank and recovering at least the oil from the algae cells.

42. The method of claim 41, wherein the first and second EMFs are created by the same apparatus.

43. The method of claim 41, wherein the first and second EMFs are created by different apparatus.
